(19) United States
(12) Patent Application Publication
(10) Pub. No.: US 2011/0245159 A1
(43) Pub. Date: Oct. 6, 2011

(54) Salts, Solvates and Pharmaceutical Compositions of Macrocyclic Ghrelin Receptor Agonists and Methods of Using the Same

(76) Inventors: Hamid R. Hoveyda, Bruxelles (BE); Martin Vézina, Quebec (CA); Eric Fournier, Laval (CA); René Gagnon, Sherbrooke (CA); Patrick Bhérer, Quebec (CA)

(21) Appl. No.: 12/937,040

(22) PCT Filed: Sep. 29, 2010
(86) PCT No.: PCT/US10/50661

§ 371 Date: Jun. 20, 2011

Related U.S. Application Data
(60) Provisional application No. 61/247,362, filed on Sep. 30, 2009.

(51) Int. Cl. A61K 38/12; C07K 5/08; C07K 1/14; C07K 1/107; C07K 1/34; A61P 1/00; A61P 1/14; A61P 1/04; A61P 1/08; A61P 29/00; A61P 3/00; A61P 5/00; A61P 9/00; A61P 25/00; A61P 19/08; A61P 35/00

(52) U.S. Cl. 514/4.9; 530/331; 514/21.1; 514/13.2; 514/17.7; 514/16.7; 514/19.2; 514/18.9

(57) ABSTRACT

The present invention provides novel salts and solvates of macrocyclic compounds that bind to and/or are functional agonists of the ghrelin (growth hormone secretagogue) receptor. The invention also relates to polymorphs of these salts and solvates, pharmaceutical compositions containing these salts or solvates, and methods of using the pharmaceutical compositions. These pharmaceutical compositions are useful as therapeutics for a range of disease indications, in particular, for treatment and prevention of gastrointestinal disorders including, but not limited to, postoperative ileus, gastroparesis, including diabetic and postsurgical gastroparesis, opioid bowel dysfunction, chronic intestinal pseudo-obstruction, short bowel syndrome, functional gastrointestinal disorders and gastrointestinal dysmotility, such as that occurring in conjunction with other disease states, in critical care situations or as a result of treatment with pharmaceutical agents. Additionally, the pharmaceutical compositions have application to the treatment and prevention of metabolic and/or endocrine disorders, cardiovascular disorders, central nervous system disorders, bone disorders, inflammatory disorders, hyperproliferative disorders, disorders characterized by apoptosis and genetic disorders.

### CROSS REFERENCE TO RELATED APPLICATIONS

This application claims priority to International Application No. PCT/US2010/050661, filed Sep. 29, 2010, which claims priority to U.S. Provisional Application Ser. No. 61/247,362, filed Sep. 30, 2009. The disclosure of each of which is incorporated herein by reference in their entireties.

### FIELD OF THE INVENTION

The present invention relates to novel salts and solvates of macrocyclic compounds that bind to and/or are functional agonists of the ghrelin (growth hormone secretagogue) receptor. The invention also relates to polymorphs of these salts and solvates, pharmaceutical compositions containing these salts or solvates, and methods of using the pharmaceutical compositions. These pharmaceutical compositions are useful as therapeutics for a range of disease indications, in particular, for treatment and prevention of gastrointestinal disorders including, but not limited to, postoperative ileus, gastroparesis, including diabetic and postsurgical gastroparesis, opioid bowel dysfunction, chronic intestinal pseudo-obstruction, short bowel syndrome, functional gastrointestinal disorders and gastrointestinal dysmotility, such as that occurring in conjunction with other disease states, in critical care situations or as a result of treatment with pharmaceutical agents. Additionally, the pharmaceutical compositions have application to the treatment and prevention of metabolic and/or endocrine disorders, cardiovascular disorders, central nervous system disorders, bone disorders, inflammatory disorders, hyperproliferative disorders, disorders characterized by apoptosis and genetic disorders.

### BACKGROUND OF THE INVENTION

Ghrelin is a recently characterized 28-amino acid peptide hormone isolated originally from the stomach of rats with the orthologue subsequently identified in humans, distinguished by an unusual n-octanoyl group modification on Ser³. (Kojima, M.; Hosoda, H. et al. Nature 1999, 402, 656-660; Kojima, M. Regul. Pept. 2008, 145, 2-6.) The existence of this hormone in a wide range of other species suggests a conserved and important role in normal physiological function. The ghrelin peptide has been demonstrated to be the endogenous ligand for a previously orphan G protein-coupled receptor (GPCR), type 1 growth hormone secretatogue receptor (GHS-R1a) (Howard, A. D.; Feighner, S. D.; et al. Science 1996, 273, 974-977; U.S. Pat. No. 6,242,199; Intl. Pat. Appl. Nos. WO 97/21730 and WO 97/22004). GHS-R1a has recently been reclassified as the ghrelin receptor (GRLN) in recognition of its endogenous ligand (Davenport, A. P.; et al. Pharmacol. Rev. 2005, 57, 541-546). GRLN is found predominantly in the brain, in particular the arcuate nucleus and ventromedial nucleus in the hypothalamus, hippocampus and substantia nigra) and pituitary, but also is expressed in a number of other tissues and organs (Gnanapavan, S.; Kola, B.; Bustin, S. A.; et al. J. Clin. Endocrinol. Metab. 2002, 87, 2988-2991; Cruz, C. R.; Smith, R. G. Vitam. Horm. 2008, 77, 47-88.).

The ghrelin peptide has been found to have a variety of endocrine and non-endocrine functions (Broglio, F.; Gottero, C.; Arvat, E.; Ghigo, E. Horm. Res. 2003, 59, 109-117; Hosoda, H.; Kojima, M.; Kangawa, K. J. Pharmacol. Sci. 2006, 100, 398-410) and this range of actions has led to the pursuit of modulators of the ghrelin receptor for a number of therapeutic purposes. (Kojima, M.; Kangawa, K. Nat. Clin. Pract. Endocrinol. Metab. 2006, 2, 80-88; Akamizu, T.; Kangawa, K. Endocr. J. 2006, 53, 585-591; Leite-Moreira, A. F.; Soares, J.-B. Drug Disc. Today 2007, 12, 276-288; Katergari, S. A.; Milousis, A.; Pagonopoulou, O.; Asimakopoulos, B.; Nikolettos, N. K. Endocr. J. 2008, 55, 439-453; Constantino, L.; Barlocca, D. Fut. Med. Chem. 2009, 1, 157-177.) For example, ghrelin and ghrelin agonists have been demonstrated to have positive effects in wasting syndromes, such as cachexia. (Kamiji, M. M.; Inui, A. Curr. Opin. Clin. Nutr. Metab. Care 2008, 11, 443-451; DeBoer, M. D. Nutrition 2008, 24, 806-814; Ashitani, J.; Matsumoto, N.; Nakazato, M. Peptides 2009, 30, 1951-1956; Cheung, W. W.; Mak, R. H. Kidney Intl. 2009, 76, 135-137.) Clinical trials have been initiated with certain of these agonists to take advantage of these effects. (Garcia, J. M.; Polyino, W. J. The Oncologist 2007, 12, 594-600; Strasser, F.; Lutz, T. A.; Maeder, M. T. Br. J. Cancer 2008, 98, 300-308; Garcia, J M.; Polyino, W. J. Growth Horm. IGF Res. 2009, 19, 267-273.) These agents also have been investigated as intervention agents in aging. (Smith, R. G.; Sun, Y.; Jiang, H.; Albarran-Zeckler, R.; Timchenko, N. Ann. N.Y. Acad. Sci. 2007, 1119, 147-164.)

As another example, the prokinetic effect of ghrelin in the gastrointestinal (GI) system makes ghrelin agonists useful for therapeutic purposes in disorders characterized by GI dysmotility. (Peeters, T. L. Curr. Opin. Pharmacol. 2006, 6, 553-558; Sanger, G. J. Drug Disc. Today 2008, 13, 234-239; Venkova, K.; Greenwood-Van Meerveld, B. Curr. Opin. Invest. Drugs 2008, 9, 1103-1107; DeSmet, B.; Mitselos, A.; Depoortere, I. Pharmacol. Ther. 2009, 123, 207-223; Camilleri, M.; Papathanasopoulos, A.; Odunsi, S. T. Nat. Rev. Gastroenterol. Hepatol. 2009, 6, 343-352; El-Salhy, M. Int. J. Mol. Med. 2009, 24, 727-732.) Such disorders include, but are not limited to, postoperative ileus, gastroparesis, including diabetic and postsurgical gastroparesis, opioid bowel dysfunction, chronic intestinal pseudo-obstruction, short bowel syndrome, functional gastrointestinal disorders and gastrointestinal dysmotility, such as that occurring in conjunction with other disease states, in critical care situations or as a result of treatment with pharmaceutical agents. Ghrelin agonists also have application as therapeutics for the treatment of cardiovascular diseases (Nagaya, N.; Kangawa, K. Drugs 2006, 66, 439-448; Garcia, E. A.; Karbonits, M. Curr. Opin. Pharmacol. 2006, 6, 142-147; Isgaard, J.; Barlind, A.; Johansson, I. Cardiovasc. Hematol. Disord. Drug Targets 2008, 8, 133-137), such as chronic heart failure, since ghrelin has been shown to be a powerful vasodilator, the treatment of bone disorders, such as osteoporosis (Svensson, J.; Lall, S.; Dickson, S. L. et al. Endocrine 2001, 14, 63-66; van der Velde, M.; Delhanty, P.; et al. Vitamins and Hormones 2007, 77, 239-258), as anti-angiogenic agents for hyperproliferative disorders, such as cancer (Baiguera, S.; Conconi, M. T.; Guidolin, D.; et al. Int. J. Mol. Med. 2004, 14, 849-854; Conconi, M. T.; Nico, B.; Guidolin, D.; et al. Peptides 2004, 25, 2179-2185), and for preventing or ameliorating conditions involving the CNS, including anxiety, stress, cognitive enhancement and sleep regulation. (Seoane, L. M.; Al-Massadi, O.; Lage, M.; Dieguez, C.; Casanueva, F. F. Pediatr. Endocrinol. Rev. 2004, 1, 432-437; McNay, E. C. Curr. Opin. Pharmacol. 2007, 7, 628-632; Ferrini, F.; Salio, C.; Lossi, L.; Merighi, A. Curr. Neuropharmacol. 2009, 7, 37-49.) Ghrelin also exhibits anti-apoptotic properties, which has been demonstrated in its ability to improve recovery after spinal cord injury (Lee, J. Y.; Chung, H.; Yoo, Y. S.; Oh, Y. J.; Oh, T. H.; Park, S, Yune, T. Y. Endocrinology. 2010, 151, 3815-3826) or after radiation exposure, such as in radiation-combined injury (Jacob, A.; Shah, K. G.; Wu, R.; Wang, P. Mol. Med. 2010, 16, 137-143), opening yet additional therapeutic potential for ghrelin receptor agonists. Lastly, ghrelin exhibits anti-inflammatory actions and, hence, ghrelin agonists can be applied to the treatment and prevention of inflammatory disorders. (Vixit, V. D.; Taub, D. D. Exp. Gerontol. 2005, 40, 900-910; Taub, D. D. Vitamins and Hormones 2007, 77, 325-346.)

A series of macrocyclic peptidomimetics recently has been described as modulators of the ghrelin receptor and their uses for the treatment and prevention of a range of medical conditions including metabolic and/or endocrine disorders, gastrointestinal disorders, cardiovascular disorders, obesity and obesity-associated disorders, central nervous system disorders, genetic disorders, hyperproliferative disorders and inflammatory disorders outlined (U.S. Pat. Nos. 7,452,862, 7,476,653 and 7,491,695; Intl. Pat. Appl. Publ. Nos. WO 2006/009645, WO 2006/009674, WO 2006/046977, WO 2006/137974 and WO 2008/130464; U.S. Pat. Appl. Publ. Nos. 2006/025566, 2007/021331, 2008/051383 and 2008/194672). The activity of one of these macrocycles, compound 298, in a rat model of POI has been reported. (Venkova, K.; Fraser, G.; Hoveyda, H. R.; Greenwood-Van Meerveld, B. Dig. Dis. Sci. 2007, 52, 2241-2248; Fraser, G. L.; Venkova, K.; Hoveyda, H. R.; Thomas, H.; Greenwood-Van Meerveld, B. Eur. J. Pharmacol. 2009, 604, 132-137.) In contrast to other types of ghrelin agonists, compound 298 did not stimulate concurrent GH secretion in these animal models. (Fraser, G. L.; Hoveyda, H. R.; Tannenbaum, G. S. Endocrinology 2008, 149, 6280-6288.) However, there remains a need for additional forms of compound 298 that are suitable for use as active pharmaceutical ingredients and appropriate for the preparation of pharmaceutical compositions.

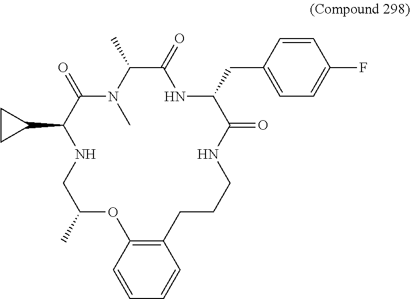

The solid state properties of an organic chemical compound are known to dramatically influence the suitability for its development as a pharmaceutical product (Berge, S. M.; Bighley, L. D.; Monkhouse, D. C. J. Pharm. Sci. 1977, 66, 1-19; Gould, P. L. Int. J. Pharm. 1986, 33, 201-217; Byrn, S. R.; Pfeiffer, R. R.; Stephenson, G.; Grant, D. J. W.; Gleason, W. B. Chem. Mater. 1994, 6, 1146-1158; Bighley, L. D.; Berge, S. M.; Monkhouse, D. C. Salt Forms of Drugs and Absorption. In Encyclopaedia of Pharmaceutical Technology; Swarbrick, J., Boylan, J. C., Eds.; Marcel Dekker, Inc.: New York, 1996; Vol. 13, pp 453-499; Stahl, P. H., Wermuth, C. G., Eds. Handbook of Pharmaceutical Salts Properties, Selection and Use; VHCA and Wiley-VCH: Zurich, Switzerland, and Weinheim, Germany, 2002; Clas, S.-D. Curr. Opin. Drug Disc. Develop. 2003, 6, 555-560; Huanga, L.-F.; Tong, W.-Q. Adv. Drug Deliv. Rev. 2004, 56, 321-334; Serajuddin, A. T. M. Adv. Drug Deliv. Rev. 2007, 59, 603-616; Paulekuhn, G. S.; Dressman, J. B.; Saal, C. J. Med. Chem. 2007, 50, 6665-6672.)

Compounds in the solid state can potentially form with one or more molecules of solvent as part of the crystalline structure, which are then termed solvates. These solvates also possess specific physicochemical and other properties that can vary significantly depending on the nature of the solvent and the number of solvent molecules associated with the crystal. Again, this can in turn greatly affect the solubility and bioavailability of the substance, as well as other pharmaceutically relevant parameters. (Vippagunta, S. R.; Brittain, H. G.; Grant, D. J. Adv. Drug Deliv. Rev. 2001, 48, 3-26.)

In addition to the identification of the most appropriate salt or solvate form, another consideration for the solid state of a substance is polymorphism. Polymorphs are different crystal forms of the identical chemical substance. (Burger, A.; Ramberger, R. Mikrochim. Acta 1979, 2, 259-271, 273-316; Vippagunta, S. R.; Brittain, H. G.; Grant, D. J. W. Adv. Drug Deliv. Rev. 2001, 48, 3-26; Singhal, D.; Curatolo, W. Adv. Drug Deliv. Rev. 2004, 56, 335-347; Llinàs, A.; Goodman, J. M. Drug Disc. Today 2008, 13, 198-210; Brittain, H. G., Ed. Polymorphism in Pharmaceutical Solids, 2^{nd} edition, Informa Healthcare, London and New York, 2009.) Different polymorphs can possess varied physical properties, including, but not limited to, melting points, solubilities, flow properties, compressibility and density dissolution rates and stability.

For macrocyclic molecules like compound 298, very little is known about their solid state behavior as few molecules of this general class have been explored as pharmaceutical products. A hydrochloride salt of compound 298, used solely as an intermediate in the purification process of compound 298, has been reported (U.S. Pat. Nos. 7,476,653; 7,491,695; and U.S. patent application Ser. No. 12/351,395), but no specific solvates or crystalline polymorphic forms were described. An unspecified formulation of compound 298 has exhibited appropriate safety and pharmacokinetic properties in humans (Lasseter, K. C.; Shaughnessy, L.; Cummings, D.; et al. J. Clin. Pharmacol. 2008, 48, 193-202), as well as efficacy for the treatment of POI (Popescu, I.; Fleshner, P. R.; Pezzullo, J. C.; Charlton, P. A.; Kosutic, G.; Senagore, A. J. Dis Colon Rectum 2010, 53, 126-134) and diabetic gastroparesis (Ejskjaer, N.; Vestergaard, E. T.; Hellstrom, P. M.; et al. Aliment Pharmacol Ther 2009, 29, 1179-1187; Ejskjaer, N.; Dimcevski, G.; Wo, J.; et al. Neurogastroenterol Motil 2010, 1069-1078).

The specific salts, solvates and polymorphic forms provided by the present invention possess superior, highly favorable properties appropriate for pharmaceutical development that were not disclosed in the prior art. Further, these solid state forms permit the preparation of pharmaceutical compositions with improved performance characteristics to be prepared.

### SUMMARY OF THE INVENTION

The present invention provides solvates of conformationally-defined macrocyclic compounds and polymorphic forms thereof. These solvates and polymorphs can function as agonists of the ghrelin (growth hormone secretagogue) receptor (GRLN, GHS-R1a) and subtypes, isoforms and variants thereof. Further, they can be readily formulated into appropriate compositions for use as pharmaceutical agents. More specifically, these solvates and polymorphs can be made reproducibly with high stability, appreciable solubility, a lack of hygroscopicity, desirable rate of dissolution and/or good bioavailability, as well as exhibiting ease in handling and in the preparation of pharmaceutical compositions.

According to aspects of the present invention, the present invention relates to solvates with the following structures:

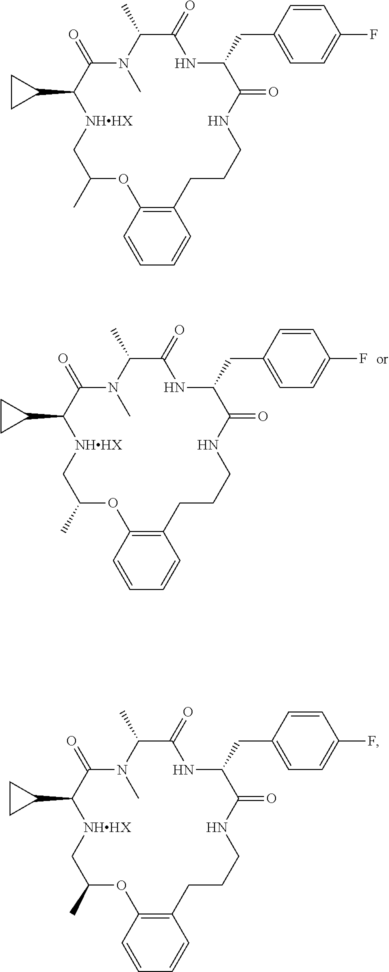

wherein HX is selected from HX hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, such as lactic acid, malic acid or tartaric acid, an amino acid, an aromatic acid and a sulfonic acid, such as methanesulfonic acid or ethanesulfonic acid.

Particular aspects of the invention provide for amorphous or crystalline forms of these solvates. Other specific aspects provide for a solvate that is a hydrate or an ethanolate.

Another particular aspect of the invention provides for the monohydrochloride monohydrate solvate, the monohydrochloride dihydrate solvate and the monhydrochloride monoethanolate solvate.

Still another particular aspect of the invention provides for polymorphic forms of solvates with the structures previously shown:

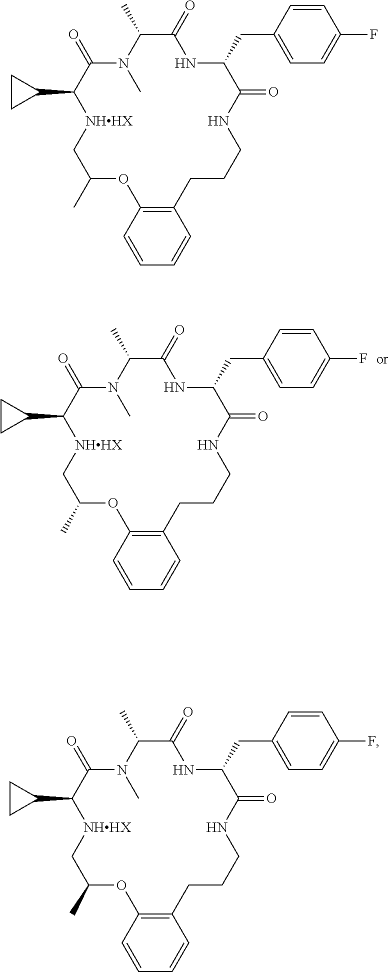

wherein HX is selected from hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, such as lactic acid, malic acid or tartaric acid, an amino acid, an aromatic acid and a sulfonic acid, such as methanesulfonic acid or ethanesulfonic acid.

In another aspect, a process for preparation of these polymorphic forms is provided. For an embodiment of the present invention, the process comprises:

(a) dissolving a macrocyclic compound with the structure

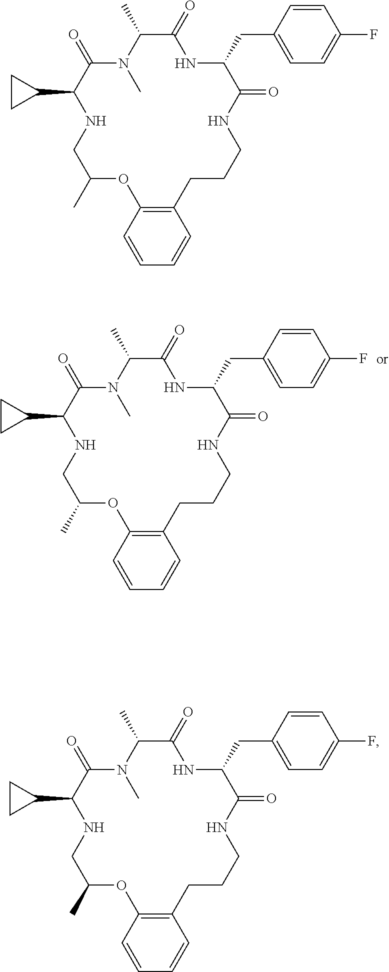

in a solution of an alcohol to form solution A;

(b) adding an acid, HX, to solution A to form acidified solution A, wherein HX is selected from hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, such as lactic acid, maliC acid or tartaric acid, an amino acid, an aromatic acid and a sulfonic acid, such as methanesulfonic acid or ethanesulfonic acid;

(c) optionally cooling acidified solution A;

(d) separating a precipitated salt from acidified solution A;

(e) dissolving the precipitated salt from (d) in a hot mixture of an alcohol and water to form solution B;

(f) cooling solution B;

(g) separating a precipitated salt from solution B;

(h) dissolving the precipitated salt from (g) in a hot mixture of a ketone solvent and water to form solution C;

(i) cooling solution C to ambient temperature or below; and

(j) separating a precipitated salt from solution C.

In certain other embodiments, the alcohol in the process is ethanol, the acid, HX, is hydrochloric acid or the ketone solvent is methyl ethyl ketone (2-butanone).

Further aspects of the present invention provide pharmaceutical compositions comprising these solvates or polymorphic forms and a pharmaceutically acceptable carrier, excipient or diluent. In some embodiments, the pharmaceutical compositions comprise (a) the polymorphic forms or solvates described herein, a buffer and a tonicity agent. In some embodiments, the pH of the acetate buffer is about 4.0 to 6.0, the acetate buffer is an acetate buffer and/or the tonicity agent is dextrose. In still some embodiments, the acetate buffer has a concentration of about 5 to 50 mM and the dextrose is present at a concentration of about 4 to 6% in water. In particular embodiments, the pharmaceutical composition comprises the polymorphic forms or solvates described herein, 10 mM acetate and 5% dextrose in water (D5W).

In certain embodiments, the solvate or polymorphic form is present in the pharmaceutical composition in an amount in a range from about 75% to about 99.9% by weight of the composition. In some embodiments, only one solvate or polymorphic form of the active substance is present during the preparation of the pharmaceutical composition and/or the final pharmaceutical composition.

In further embodiments, the pharmaceutical composition is a solid dosage form. In still further embodiments, the pharmaceutical compositions is an aqueous dosage form, i.e., provided in a solvent.

In a particular aspect, a buffered aqueous pharmaceutical composition of the monohydrochloride monohydrate is provided.

Another particular aspect provides a process for preparation of these pharmaceutical compositions. In an embodiment of the invention wherein the pharmaceutically acceptable carrier, excipient or diluent is a buffer and a tonicity agent, the process comprises:

(a) dissolving a tonicity agent in solvent to form solution D;

(b) adding acid to solution D to form acidic solution D;

(c) dissolving a macrocyclic compound with the structure

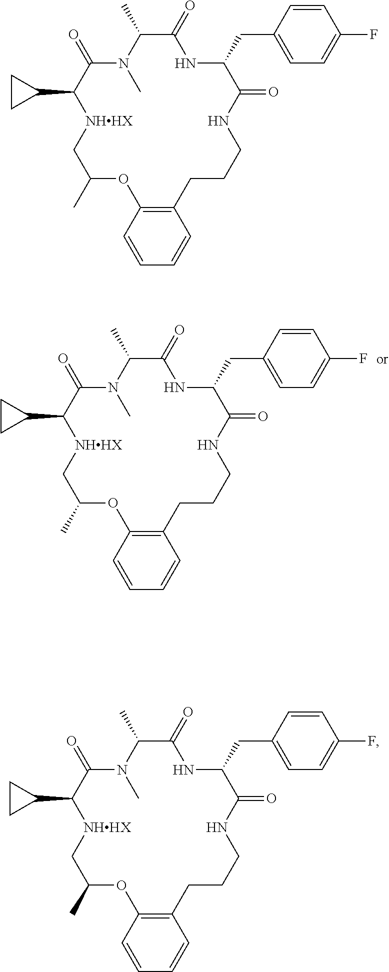

wherein HX is selected from hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, such as lactic acid, malic acid or tartaric acid, an amino acid, an aromatic acid and a sulfonic acid, such as methanesulfonic acid or ethanesulfonic acid; in acidified solution D to form solution E;

(d) adjusting the pH of solution E through the addition of base to form solution F; and

(e) diluting solution F with solvent to an effective concentration.

In an embodiment, the steps in the preceding process are conducted sequentially while in another embodiment, the steps are conducted in the order step (b), then step (d), then step (a), then step (c), then step (e).

In still other embodiments, the solvent of this process is water for injection, the tonicity agent is dextrose, the acid is acetic acid, the base is sodium hydroxide, the pH is adjusted to between 4.0-5.0, or the effective concentration is 0.05-5.0 mg/mL. In specific embodiments of this process, the pH is between 4.3-4.7 or the effective concentration is 1.0±0.1 mg/mL or 2.0±0.2 mg/mL given as free base equivalents. In an additional embodiment, the process further comprises filtration though one or more sterilizing filters, such as 0.22 μm filters.

In another aspect of the invention, salts of macrocyclic compounds have the following structure:

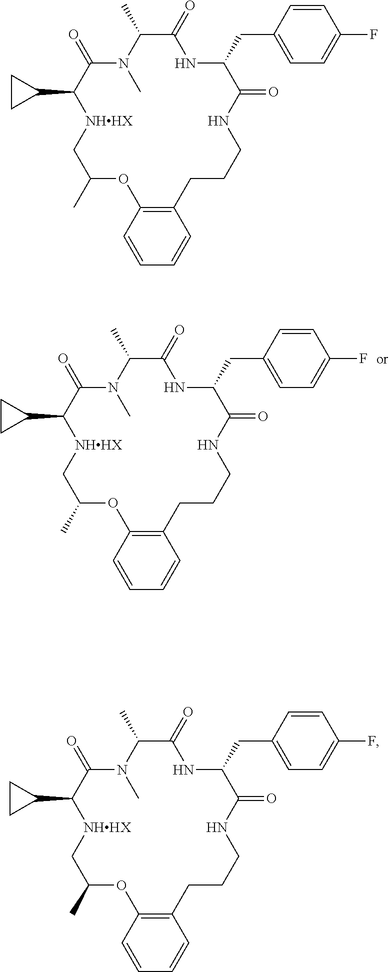

wherein HX is selected from carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, such as lactic acid, malic acid or tartaric acid, an amino acid, an aromatic acid and a sulfonic acid, such as methanesulfonic acid or ethanesulfonic acid, are provided.

Further aspects of the present invention relate to methods of making the salts, solvates and polymorphs and pharmaceutical compositions thereof.

Other aspects of the present invention provide methods of treating a gastrointestinal disorder, a disorder characterized by reduced appetite or decreased food intake, a metabolic or endocrine disorder, a cardiovascular disorder, an inflammatory disorder, a bone disorder, a disorder characterized by apoptosis or a hyperproliferative disorder, with an effective amount of a pharmaceutical composition containing the solvates or polymorphic forms.

Additional aspects of the present invention further provide methods of stimulating gastrointestinal motility, and/or treating a gastrointestinal disorder comprising administering to a subject an effective amount of these salts, solvates or polymorphic that stimulates a mammalian GRLN receptor.

Aspects of the present invention further relate to methods of preventing and/or treating disorders described herein, in particular, gastrointestinal disorders, including post-operative ileus, gastroparesis, such as diabetic and post-surgical gastroparesis, opioid-induced bowel dysfunction, chronic intestinal pseudo-obstruction, short bowel syndrome, functional gastrointestinal disorders, gastrointestinal dysmotility, such as that occurring in conjunction with other disease states, including infections, neurological diseases, neuromuscular conditions, connective tissue diseases, and endocrine or metabolic disturbances, in critical care situations or as a result of treatment with pharmaceutical agents, emesis such as caused by cancer chemotherapy, constipation such as associated with the hypomotility phase of irritable bowel syndrome (IBS), delayed gastric emptying associated with wasting conditions, gastroesophageal reflux disease (GERD), gastric ulcers, Crohn's disease and other diseases and disorders of the gastrointestinal tract.

In particular embodiments, the gastrointestinal disorder is postoperative ileus, gastroparesis, diabetic gastroparesis, postsurgical gastroparesis, opioid-induced bowel dysfunction, chronic intestinal pseudo-obstruction, acute colonic pseudo-obstruction (Ogilvie's syndrome), short bowel syndrome, emesis, constipation-predominant irritable bowel syndrome (IBS), chronic constipation, functional dyspepsia, cancer-associated dyspepsia syndrome, graft versus host disease, gastroesophageal reflux disease (GERD), gastric ulcers, Crohn's disease, gastroenteritis, gastrointestinal dysfunction or delayed gastric emptying in patients with eating disorders, including anorexia nervosa and bulimia, gastrointestinal dysfunction or delayed gastric emptying in patients with Parkinson's disease, gastrointestinal dysfunction or delayed gastric emptying in patients with myotonic muscular dystrophy, gastrointestinal dysfunction or delayed gastric emptying in patients with autonomic degeneration, gastrointestinal dysfunction or delayed gastric emptying in patients who have suffered a stroke, gastrointestinal dysfunction or delayed gastric emptying in patients with multiple sclerosis, gastrointestinal dysfunction or delayed gastric emptying in patients with neurological diseases and disorders, including amyloid neuropathy, primary dysautonomia, vagal injury and pyloric stenosis, gastrointestinal dysfunction or delayed gastric emptying in patients with psychiatric diseases, including depression, gastrointestinal dysfunction or delayed gastric emptying in patients with scleroderma, gastrointestinal dysfunction or delayed gastric emptying in patients with cystic fibrosis, gastrointestinal dysfunction or delayed gastric emptying in patients with connective tissue diseases, including, systemic sclerosis, dermatomyositis, polymyositis, systemic lupus erythematosis and amyloidosis, gastrointestinal dysfunction or delayed gastric emptying in patients with liver cirrhosis, gastrointestinal dysfunction or delayed gastric emptying in patients with liver failure, gastrointestinal dysfunction or delayed gastric emptying in patients with renal failure, gastrointestinal dysfunction or delayed gastric emptying in patients with gallbladder disorders, gastrointestinal dysfunction or delayed gastric emptying in patients with migraines, gastrointestinal dysfunction or delayed gastric emptying with sepsis, gastrointestinal dysfunction or delayed gastric emptying in patients with brain stem lesions, gastrointestinal dysfunction or delayed gastric emptying in patients with spinal cord injury, gastrointestinal dysfunction or delayed gastric emptying in patients with cancer, including stomach, biliary, esophageal, gastric and pancreatic cancers, gastrointestinal dysfunction or delayed gastric emptying in patients with neoplasia, gastrointestinal dysfunction or delayed gastric emptying in patients who have undergone radiation treatment, gastrointestinal dysfunction or delayed gastric emptying in patients with achalasia, gastrointestinal dysfunction or delayed gastric emptying in patients with infectious diseases, including HIV, Herpes zoster infection and Chagas disease, gastrointestinal dysfunction or delayed gastric emptying as a result of surgery, gastrointestinal dysfunction or delayed gastric emptying in patients with critical illness, gastrointestinal dysfunction or delayed gastric emptying in patients requiring critical care, gastrointestinal dysfunction or delayed gastric emptying in patients after transplants, including heart or lung transplantation, gastrointestinal dysfunction or delayed gastric emptying in patients with Turner's syndrome, gastrointestinal dysfunction or delayed gastric emptying as a result of treatment with pharmaceutical agents, including opioids, anticholinergics, beta blockers, calcium channel antagonists, glucagon-like peptide-1 (GLP-1) receptor agonists, amylin receptor agonists, peptide YY (PYY) receptor agonists, proteasome inhibitors, tricyclic antidepressants, monoamine uptake blocker antidepressants, cancer chemotherapy agents, adrenergic agonists, dopaminergic agents, antimalarials, antispasmodics, cannabinoid agonists, octreotide, levodopa, alcohol and nicotine, gastrointestinal dysfunction or delayed gastric emptying as a result of endocrine disturbances, including hypothyroidism, hyperthyroidism, Addison's disease and porphyria, gastrointestinal dysfunction or delayed gastric emptying as a result of metabolic disturbances, including hyperglycemia, hypokalemia and hypomagnesemia, gastrointestinal dysfunction or delayed gastric emptying as a result of anesthesia, gastrointestinal dysfunction or delayed gastric emptying as a result of mechanical ventilation, gastrointestinal dysfunction or delayed gastric emptying as a result of electrolyte disturbances, gastrointestinal dysfunction or delayed gastric emptying as a result of severe trauma or gastrointestinal dysfunction or delayed gastric emptying as a result of pain.

The present invention also relates to solvates or polymorphic forms used for the preparation of a medicament for prevention and/or treatment of the disorders described herein.

Still other aspects of the present invention provide kits comprising one or more containers containing pharmaceutical dosage units comprising an effective amount of one or more compounds of the present invention packaged with optional instructions for the use thereof.

The foregoing and other aspects of the present invention are explained in greater detail in the specification set forth below.

### BRIEF DESCRIPTION OF THE DRAWINGS

FIG. 1 shows a synthetic route to a representative solvate of the invention compound 298.HCl.H₂O.

FIG. 2 shows a single crystal X-ray structure of a representative solvate of the invention, compound 298.HCl.H₂O.

FIG. 3 shows a single crystal X-ray structure of another representative solvate of the invention, compound 298.HCl.2H₂O.

FIG. 4 shows a single crystal X-ray structure of another representative solvate of the invention, compound 298.HCl.EtOH.

FIG. 5 shows a ¹H NMR spectrum of a representative solvate of the invention, compound 298.HCl.H₂O.

FIG. 6 shows a ¹³C NMR spectrum of a representative solvate of the invention, compound 298.HCl.H₂O.

FIG. 7 shows an ¹⁹F NMR spectrum of a representative solvate of the invention, compound 298.HCl.H₂O.

FIG. 8 shows am FT-IR spectrum of a representative solvate of the invention, compound 298.HCl.H₂O.

FIG. 9 shows an X-ray powder diffractogram (XRPD) of a representative polymorphic form of the invention.

FIG. 10 shows a differential scanning calorimetry (DSC) thermogram of a representative solvate of the invention, compound 298.HCl.H₂O.

FIG. 11 shows results of a dynamic vapor sorption/desorption (DVS) experiment for a representative solvate of the invention, compound 298.HCl.H₂O.

### DETAILED DESCRIPTION

The foregoing and other aspects of the present invention will now be described in more detail with respect to the embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

A “stable compound” or “stable structure” refers to a compound that is sufficiently robust to survive isolation to a useful degree of purity and formulation into an efficacious therapeutic agent.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “unnatural” or “unusual” refers to the wide selection of non-natural, rare or synthetic amino acids such as those described in the literature by Hunt, S. in Chemistry and Biochemistry of the Amino Acids, Barrett, G. C., Ed., Chapman and Hall: New York, 1985; Kamphuis, J.; Meijer, E. M.; Boesten, W. H.; et al. Ann. N.Y. Acad. Sci. 1992, 672, 510-527; Kotha, S. Acc. Chem. Res. 2003, 36, 342-351; Cardillo, G.; Gentilucci, L.; Tolomelli, A. Mini-Rev. Med. Chem. 2006, 6, 293-304; Fotheringham, I.; Archer, I.; Carr, R.; Speight, R.; Turner, N. J. Biochem. Soc. Trans. 2006, 34, 287-290.

Abbreviations used for amino acids and designation of peptides follow the rules of the IUPAC-IUB Commission of Biochemical Nomenclature in J. Biol. Chem. 1972, 247, 977-983. This document has been updated: Biochem. J. 1984, 219, 345-373; Eur. J. Biochem. 1984, 138, 9-37; 1985, 152, 1; Internat. J. Pept. Prot. Res. 1984, 24, following p 84; J. Biol. Chem. 1985, 260, 14-42; Pure Appl. Chem. 1984, 56, 595-624; Amino Acids and Peptides 1985, 16, 387-410; and in Biochemical Nomenclature and Related Documents, 2nd edition, Portland Press, 1992, pp 39-67. Extensions to the rules were published in the JCBN/NC-IUB Newsletter 1985, 1986, 1989; see Biochemical Nomenclature and Related Documents, 2nd edition, Portland Press, 1992, pp 68-69.

The term “agonist” refers to a compound that duplicates at least some of the effect of the endogenous ligand of a protein, receptor, enzyme or the like.

The term “effective amount” or “effective” is intended to designate a dose that causes a relief of symptoms of a disease or disorder as noted through clinical testing and evaluation, patient observation, and/or the like, and/or a dose that causes a detectable change in biological or chemical activity. The detectable changes may be detected and/or further quantified by one skilled in the art for the relevant mechanism or process. As is generally understood in the art, the dosage will vary depending on the administration routes, symptoms and body weight of the patient but also depending upon the compound being administered.

Administration of two or more compounds “in combination” means that the two compounds are administered closely enough in time that the presence of one alters the biological effects of the other. The two compounds can be administered simultaneously (concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “pharmaceutically acceptable salt” is intended to mean a salt form of a compound that permits its use or formulation as a pharmaceutical and which retains the biological effectiveness of the specified compound and that is not biologically or otherwise undesirable. Some such salts are described in Stahl, P. H., Wermuth, C. G., Eds. Handbook of Pharmaceutical Salts: Properties, Selection and Use; VHCA and Wiley-VCH: Zurich, Switzerland, and Weinheim, Germany, 2002.

The term “pharmaceutically active metabolite” is intended to mean a pharmacologically active product produced through metabolism in the body of a specified compound.

The term “salt” is intended to mean an ionic compound produced from contacting an acid and a base. Salts can be amorphous, crystalline or partially crystalline when in solid form.

The term “solvate” is intended to mean a pharmaceutically acceptable solid form of a specified compound containing solvent molecules as part of the crystal structure. A solvate typically retains at least some of the biological effectiveness of such compound. Solvates can have different solubilities, hygroscopicities, stabilities and other properties. Examples of solvates, without limitation, include compounds in combination with water, isopropanol, ethanol, methanol, DMSO, ethyl acetate, acetic acid, or ethanolamine. Solvates are sometimes termed “pseudopolymorphs.”

The term “hydrate” is intended to mean a solvate with water.

The term “ethanolate” is intended to mean a solvate with ethanol.

The term “polymorph” or “polymorphic form” is intended to mean a single crystalline form of a material. A crystalline material may have one or more polymorphic forms. Polymorphs have the same chemical composition, but different arrangements or conformations of the molecules in the crystal lattice structures. Different polymorphs can possess different physical and chemical properties, including different densities, melting points, solubilities and other properties.

### 1. Compounds

The compounds disclosed herein may have asymmetric centers. The inventive compounds may exist as single stereoisomers, racemates, and/or mixtures of enantiomers and/or diastereomers. All such single stereoisomers, racemates, and mixtures thereof are intended to be within the scope of the present invention. In particular embodiments, however, the inventive compounds are used in optically pure form. The terms “S” and “R” configuration as used herein are as defined by the IUPAC 1974 Recommendations for Section E, Fundamentals of Stereochemistry (Pure Appl. Chem. 1976, 45, 13-30). Unless otherwise depicted to be a specific orientation, the present invention accounts for all stereoisomeric forms.

As generally understood by those skilled in the art, an “optically pure” compound is one that contains only a single enantiomer. As used herein, the term “optically active” is intended to mean a compound comprising at least a sufficient excess of one enantiomer over the other such that the mixture rotates plane polarized light. Optically active compounds have the ability to rotate the plane of polarized light. The excess of one enantiomer over another is typically expressed as enantiomeric excess (e.e.). In describing an optically active compound, the prefixes D and L or R and S are used to denote the absolute configuration of the molecule about its chiral center(s). The prefixes “d” and “l” or (+) and (−) are used to denote the optical rotation of the compound (i.e., the direction in which a plane of polarized light is rotated by the optically active compound). The “l” or (−) prefix indicates that the compound is levorotatory (i.e., rotates the plane of polarized light to the left or counterclockwise) while the “d” or (+) prefix means that the compound is dextrorotatory (i.e., rotates the plane of polarized light to the right or clockwise). The sign of optical rotation, (−) and (+), is not related to the absolute configuration of the molecule, R and S.

A compound of the invention having the desired pharmacological properties will be optically active and, can be comprised of at least 90% (80% e.e.), at least 95% (90% e.e.), at least 97.5% (95% e.e.) or at least 99% (98% e.e.) of a single isomer.

The salts, solvates and/or polymorphs of the present invention show increased stability in comparison to the previously known compounds. The stability under various environmental conditions, in particular, can ensure that no decomposition products with potentially undesirable side effects are formed and that the amount of active substance in a pharmaceutical composition is not reduced below an effective amount over time or storage. Similarly, the substance must remain stable during the necessary processing involved in the preparation of a pharmaceutical composition containing that substance.

The salts, solvates and/or polymorphs of the present invention show increased solubility of the active substance. This is desirable in cases where, for example, during preparation of a pharmaceutical composition in solution, such as for injection or infusion, the active substance must be sufficiently soluble in a physiologically acceptable solvent and remain soluble over time and storage. Similarly, for an oral formulation, the active substance also must be sufficiently soluble in physiological fluid so that the rate of dissolution after administration permits therapeutic levels of the active substance to be reached in the plasma. The salts, solvates and/or polymorphs of the present invention can possess these capabilities.

For the preparation of pharmaceutical compositions for oral administration, the solid state properties of an active substance are beneficial for other reasons as well. Flowability affects the ease with which the substance can be handled during the manufacturing and processing of the pharmaceutical composition, typically a tablet or capsule, although this also pertains to preparation of a liquid composition like a syrup or elixir. Poor flowability typically requires the addition of excipients in order to improve the flow properties, which increases the complexity and cost of the pharmaceutical composition. (Aleeva, G. N.; Zhuravleva, M. V.; Khafizyanova, R. K. Pharm. Chem. J. 2009, 43, 230-234.) The solid state form impacts the compressibility of the active substance, an important parameter for solid dosage formulations, as well. The salts, solvates and/or polymorphs of the present invention can possess these capabilities.

The hygroscopicity of an active substance is also a parameter of interest. A pharmaceutical substance that absorbs moisture increases weight and thereby reduces the relative content of the active component. Such substances are generally specially stored to prevent such uptake of moisture. Hygroscopicity also can create difficulties during the preparation of the active substance or pharmaceutical compositions containing it as the uptake of moisture during manufacturing can cause technical issues with processing and isolation procedures. The salts, solvates and/or polymorphs of the present invention can exhibit low hygroscopicity.

### 2. Synthetic Methods

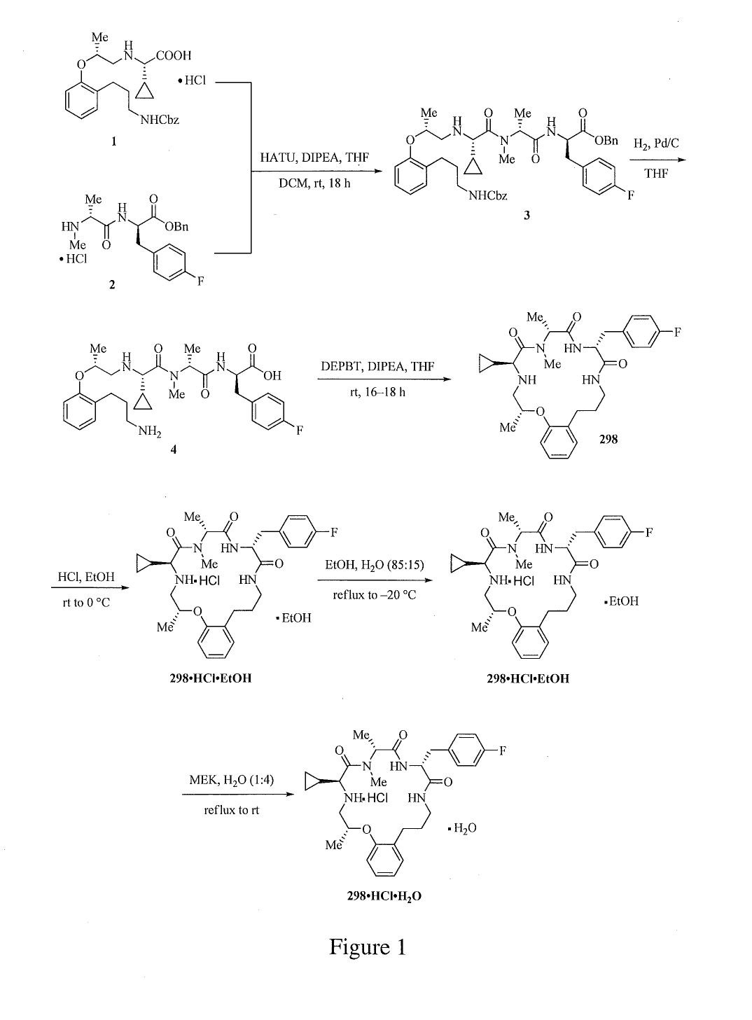

Synthetic methods for the general type of macrocyclic structure used for the salts, solvates and polymorphs of the present invention are described in Intl. Pat. Appls. WO 01/25257, WO 2004/111077, WO 2005/012331 and WO 2005/012332. Compound 298 and its hydrochloride salt are prepared as outlined in FIG. 1 (U.S. Pat. Nos. 7,476,653 and 7,491,164; U.S. Patent Appl. Publ. 2009/0198050; and U.S. patent application Ser. No. 12/351,395). The salts, solvates and polymorphs of the present invention can be prepared using the general methods described below as well as those provided in the Examples.

Method 2A. General Method for the Preparation of Representative Salts or Solvates of the Invention

The following general procedure was employed to prepare representative salts or solvates of the invention:
(a) one equivalent (1.0 eq) of the macrocyclic compound as its fee base is added to an appropriate container;
(b) to the free base is added 1.1 eq of an aqueous solution of the acid;
(c) the resulting mixture is agitated for up to 72 hr;
(d) the mixture is heated and an organic solvent added (using such techniques as dropwise or as a fixed ratio to the aqueous volume);
(e) the hot mixture is permitted to slowly cool to room temperature, with optional cooling further to 4° C.;
(f) the precipitated salt is collected by filtration and washed.

Method 2B. General Method for the Preparation of a Representative Polymorph of the Invention

The following procedure can be employed to prepare a representative polymorphic form of the invention:
(a) dissolve the macrocyclic compound in a solution of an alcohol to form solution A;
(b) add an acid to solution A to form acidified solution A, which can then be optionally cooled;
(c) separate the precipitated salt from acidified solution A;
(d) dissolve the precipitated salt from (c) in a hot mixture of an alcohol and water to form solution B;
(e) cool solution B;
(f) separate the precipitated salt from solution B;
(g) dissolve the precipitated salt from (f) in a hot mixture of a ketone solvent and water to form solution C;
(h) cool solution C to ambient temperature or below; and
(i) separate the precipitated polymorphic form from solution C.

A salt of the invention also may be prepared by any suitable method known to those skilled in the art, including treatment of the free base with an inorganic acid, such as hydrochloric acid, hydrobromic acid, hydroiodic acid, carbonic acid, sulfuric acid, nitric acid, phosphoric acid, and the like, or with an organic acid, including formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, pyranosidyl acid, such as glucuronic acid or galacturonic acid, alpha-hydroxy acid, such as lactic acid, malic acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, 2-hydroxyethanesulfonic acid, benzenesulfonic acid, cyclohexyl-aminosulfonic acid or the like. The preparations of representative salts of the invention are provided in the Examples.

Salts of the invention can form solvates with certain solvents in which they come into contact. These solvents are typically those involved in reactions or purifications of the compounds. Representative salts, solvates and polymorphs of the invention are prepared as described in the Examples.

### 3. Pharmaceutical Compositions

The salts, solvates and polymorphs of the present invention may be formulated into pharmaceutical compositions of various dosage forms. The salts, solvates and polymorphs of the present invention may be included in the various dosage forms in an amount from about 75%, 80%, 85%, 90%, 95%, 99% or 99.9%. Thus, a particular dosage form of the pharmaceutical composition may include a controlled, stable and/or desired amount of the salt form, solvate form or polymorphic form of the compounds described herein. In some embodiments, the most thermodynamically stable polymorphic form of the active substance is included in the dosage form.

To prepare the pharmaceutical compositions of the invention, one or more salts, solvates or polymorphs as the active ingredient(s) is intimately mixed with appropriate carriers, excipients and additives according to techniques known to those skilled in the art of pharmaceutical formulations.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like, with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation that also may be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral or infusion (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (WFI, USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the solvates, polymorphs and salts of the macrocyclic compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In some embodiments, the composition is provided in a unit dosage form such as a tablet or capsule.

In further embodiments, the present invention provides kits including one or more containers comprising pharmaceutical dosage units comprising an effective amount of one or more salts, solvates or polymorphs of the present invention.

In specific embodiments, the kits contain vials or syringes comprising pharmaceutical dosage units comprising an effective amount of one or more salts, solvates or polymorphs of the present invention.

The present invention further provides that the solvates, salts and polymorphs of the present invention may be administered in combination with a therapeutic agent used to prevent and/or treat metabolic and/or endocrine disorders, gastrointestinal disorders, cardiovascular disorders, obesity and obesity-associated disorders, central nervous system disorders, bone disorders, genetic disorders, hyperproliferative disorders, disorders characterized by apoptosis and inflammatory disorders. Exemplary agents include analgesics (including opioid analgesics), anesthetics, antifungals, antibiotics, antiinflammatories (including nonsteroidal anti-inflammatory agents), anthelmintics, antiemetics, antihistamines, antihypertensives, antipsychotics, antiarthritics, antitussives, antivirals, cardioactive drugs, cathartics, chemotherapeutic agents (such as DNA-interactive agents, antimetabolites, tubulin-interactive agents, hormonal agents, and agents such as asparaginase or hydroxyurea), corticoids (steroids), antidepressants, depressants, diuretics, hypnotics, minerals, nutritional supplements, parasympathomimetics, hormones (such as corticotrophin releasing hormone, adrenocorticotropin, growth hormone releasing hormone, growth hormone, thyrptropin-releasing hormone and thyroid stimulating hormone), sedatives, sulfonamides, stimulants, sympathomimetics, tranquilizers, vasoconstrictors, vasodilators, vitamins and xanthine derivatives.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes.

In therapeutic use for treatment of conditions in mammals (i.e. humans or animals) for which an agonist of the ghrelin receptor is effective, the solvates, salts or polymorphs of the present invention or an appropriate pharmaceutical composition thereof may be administered in an effective amount. Since the activity of the materials and the degree of the therapeutic effect vary, the actual dosage administered will be determined based upon generally recognized factors such as age, condition of the subject, route of delivery and body weight of the subject. The dosage can be from about 0.1 to about 100 mg/kg, administered orally 1-4 times per day. In addition, solvates, salts or polymorphs in an appropriate pharmaceutical composition can be administered by injection at approximately 0.01-20 mg/kg per dose, with administration 1-4 times per day. Treatment could continue for weeks, months or longer. Thus, treatment can be acute or chronic. Determination of optimal dosages for a particular situation is within the capabilities of those skilled in the art.

Method 3A. General Method for the Preparation of a Representative Pharmaceutical Composition of the Invention

The following procedure can be used to prepare a representative formulation containing salts, solvates or polymorphs of the invention.
(a) dissolve a tonicity agent, for example saline solution or 5% dextrose in water, in solvent, such as water for injection, to form a new solution D;
(b) add an acid, such as acetic acid, to form acidified solution D;
(c) dissolve the salt, solvate or polymorph in acidified solution D to form solution E;
(d) adjust the pH of solution E through the addition of base, for example sodium hydroxide, to form solution F; and
(e) dilute solution F with solvent to an effective concentration.

Certain representative pharmaceutical compositions of the invention are presented in the Examples.

### 4. Analytical Methods

The identity and characterization of solvates, salts and polymorphs of the invention can be done utilizing a series of well-established analytical and physicochemical techniques. In some cases, specific methods for these techniques have been developed for the solvates, salts and polymorphs of the invention. Certain standard analysis methods are provided in the United States Pharmacopeia-National Formulary (USP-NF), a book of public pharmacopeial standards that contains standards for medicines, dosage forms, drug substances, excipients, medical devices and dietary supplements. Such methods are denoted by USP <#>, where # indicates the applicable numerical chapter in the USP-NF.

In addition to the use of single-crystal X-ray diffraction to provide structural information about a crystalline solid form, a number of different analytical techniques have been developed to quantitate and differentiate between amorphous and crystalline forms, as well as between crystalline and polymorphic forms (Bugay, D. E. Adv. Drug Deliv. Rev. 2001, 48, 43-65; Shah, B.; Kakumanu, K.; Bansal, A. K. J. Pharm. Sci. 2006, 95, 1641-1665.) These include X-ray powder diffraction (XRPD), gravimetric thermal analysis, differential scanning calorimetry (DSC), solution microcalorimetry, isothermal microcalorimetry (IMC), Raman spectroscopy, near infrared spectroscopy (NIR), diffuse reflectance infrared (IR) spectroscopy, attenuated reflectance spectroscopy, solid state nuclear magnetic resonance (NMR), dynamic vapor sorption (DVS), terahertz pulsed spectroscopy (TPS), thermally stimulated current spectroscopy (TSC), dynamic mechanical analysis (DMA) and inverse gas chromatography (IGC).

Different polymorphic forms can be distinguished by their thermal behavior and can be separately characterized using methods such as melting point, thermogravimetric analysis (TGA) and DSC. A specific polymorphic form possesses distinct spectroscopic properties that can be detected using techniques such as XRPD, solid state ¹³C NMR spectrometry and IR spectroscopy.

For determining the stability of an active pharmaceutical ingredient (API) or a pharmaceutical composition, the approach as outlined for regulatory purposes in International Conference on Harmonisation of Technical Requirements for Registration of Pharmaceuticals for Human Use (ICH) Q1A Guideline: Stability Testing of New Drug Substances and Products, February 2003, can be followed. In summary, this entails repetition of certain analytical tests on samples stored at three representative controlled conditions to ascertain whether or not any degradation or loss of potency has occurred. The typical conditions employed are: (1) 25° C.±2° C., 60%±5% relative humidity (RH); (2) 30° C.±2° C., 65%±5% RH; (3) 40° C.±2° C., 75%±5% RH (often termed accelerated stability). The photostability as outlined in International Conference on Harmonisation of Technical Requirements for Registration of Pharmaceuticals for Human Use (ICH) Q1B Guideline: Stability Testing: Photostability Testing of New Drug Substances and Products, November 1996, is typically also investigated.

The following general methods can be employed to characterize the salts, solvates or polymorphs of the invention.

Method 4A. Appearance

Examine the sample visually and report the physical state and color.

Method 4B. HPLC Assay—Purity, Impurities and Potency

The purpose of this procedure is to determine the purity of representative salts, solvates and polymorphs of the invention by reverse phase HPLC. Any appropriate detection method compatible with HPLC can be utilized, such as single or dual wavelength ultraviolet (UV) detection, evaporative light scattering detection (ELSD) or chemiluminescent nitrogen detection (CLND). For most cases, UV detection would be preferred. Purity can be determined by peak area %. The same assay conditions are used to determine the level of impurities and the potency. The parameters for this HPLC assay are delineated below.

Mobile Phase A: 10 mM Ammonium Hydroxide in Water
Add 1.8 mL of ammonium hydroxide (21%) to 2 L of water and mix thoroughly. If required by the HPLC system, the solvent can be degassed by an appropriate method such as an online degasser or He sparging.

Mobile Phase B: 10 mM Ammonium Hydroxide in Acetonitrile
Add 1.8 mL of ammonium hydroxide (21%) to 2 L of acetonitrile and mix thoroughly. If required by the HPLC system, the solvent can be degassed by an appropriate method such as an online degasser or He sparging.

Diluent: Water/Acetonitrile (1/1, v/v)
Add 500 mL acetonitrile and 500 mL water to an appropriate container and mix thoroughly. This diluent is typically also used for blank sample preparations.

Typical Chromatographic Conditions
Column: XTerra RP18, 3.5 μm, 4.6×100 mm (or equivalent)
Detection: 230 nm
Column Temperature: 30° C.
Injection Volume: 10 μL
Flow Rate: 1 mL/min
Run Time: 46.0 min
Data Acquisition Time: 37.5 min
Mobile Phase A: 10 mM Ammonium Hydroxide in Water
Mobile Phase B: 10 mM Ammonium Hydroxide in Acetonitrile

Gradient

| Time (min) | % A | % B | Flow Rate (mL/min) |
|---|---|---|---|
| 0.00 | 80.0 | 20.0 | 1.0 |
| 25.00 | 50.0 | 50.0 | 1.0 |
| 35.00 | 0.0 | 100.0 | 1.0 |
| 37.50 | 0.0 | 100.0 | 1.0 |
| 38.00 | 80.0 | 20.0 | 1.0 |
| 46.00 | 80.0 | 20.0 | 1.0 |

Blank Preparation:
Use water/acetonitrile (1/1) as blank.

Sample Preparation
Weigh approximately 25 mg of sample and dissolve/dilute to 50.0 mL with water/acetonitrile (1/1) (or an appropriate solution to provide a concentration of 0.5 mg/mL).

Standard Preparation
To confirm identity or determine potency for known compounds, weigh an appropriate amount of the standard sample into a 50 mL volumetric flask. Dissolve in water/acetonitrile (1/1) (or other appropriate solvent), dilute to volume and mix thoroughly. Samples at different concentrations can be prepared by accurate dilution of specific volume quantities of the standard sample. Such samples can also be used for system suitability confirmation.

Sample Analysis Procedure
Equilibrate the chromatographic system.
Inject the blank and confirm there are no significant interferences at the expected retention times of the peaks of interest.
Inject the standard (or series of standards as required for the analysis).
Inject each sample preparation once.
Insert appropriate standard samples during analysis of multiple samples.
Measure the % area response of all peaks in the sample injection which are equal to or above the limit of quantitation (0.05%).

Alternative Chromatographic Conditions
Column: XTerra RP18, 3.5 μm, 4.6×100 mm (or equivalent)
Detection: 225 nm
Column Temperature: 30° C.
Injection Volume: 20 μL
Flow Rate: 1.2 mL/min
Run Time: 46.0 min
Re-Equilibration Time; 8.5 min
Data Collection Time: 37.5 min
Needle Wash: 50/50 Acetonitrile/Water (v/v)
Mobile Phase A: 10 mM Ammonium Hydroxide in Water
Mobile Phase B: 10 mM Ammonium Hydroxide in Acetonitrile

Gradient

| Time (min) | % A | % B |
|---|---|---|
| 0.0 | 80 | 20 |
| 0.5 | 80 | 20 |
| 27.0 | 60 | 40 |
| 35.0 | 0 | 100 |
| 37.5 | 0 | 100 |
| 38.0 | 80 | 20 |
| 46.0 | 80 | 20 |

Method 4C. Analysis by UV-Visible Spectroscopy

The ultraviolet spectrum can be obtained using an Ultrospec 2100 Pro UV/Vis spectrophotometer (or similar) for an appropriate solution of a salt, solvate or polymorph of the invention.

Method 4D. Analysis of Identity by ¹H NMR, ¹³C NMR and ¹⁹F NMR

¹H, ¹³C and ¹⁹F NMR spectra are recorded in accordance with USP <761> using a Varian Mercury VX-300 MHz spectrometer, a Bruker Avance 300 MHz spectrometer or a Bruker Avance 500 MHz spectrometer (or similar). Spectra were typically analyzed for consistency of the sample with a standard for the structure.

Method 4E. Analysis by Fourier-Transform Infrared (FT-IR)

The Fourier-transformed infrared (FT-IR) absorption spectrum of salts, solvates and polymorphs of the invention can be obtained using a Perkin Elmer 1600 FT-IR spectrometer (or similar) in an appropriate solution or as a potassium bromide (KBr) pellet.

Method 4F. Determination of Chloride Ion

The sample is dissolved in acetonitrile:water (1:1), then diluted with water and 6 N nitric acid. Quantification of chloride ion is then determined by potentiometric titration using a silver nitrate solution.

An alternative method can be employed using oxygen combustion followed by potentiometric titration.

Method 4G. Determination of Moisture Level

Moisture level is determined by Karl Fischer titrimetry using the direct titration method, in accordance with USP <921 1a>.

Method 4H. Residue on Ignition

Residue on ignition is determined by the sulfated ash test in accordance with USP <281>.

Method 41. Determination of Endotoxin Levels

Endotoxin levels are measured using the gel clot method in accordance with USP <85>.

Method 4J. Determination of Bioburden

Bioburden is assessed by total bacterial count as well as total yeast and mold count in accordance with USP <61>.

Method 4K. X-Ray Powder Diffraction (XRPD) Analysis

For evaluation of the crystallinity and polymorphic forms of salts and solvates of the present invention, XRPD can be conducted in a wide-angle powder X-ray diffractometer (Siemens D5005, Shimadzu Model XRD-6000 or similar) operating under ambient conditions (22°±3° C.). This typically is performed in a step-scan mode, in increments of 0.05° 2θ, from 5 to 40° 2θ and the counts were accumulated for 1 sec at each step. The milled powder sample or other appropriately prepared sample can be top-filled into an aluminum holder and exposed to Cu K_{α} radiation.

Method 4L. Differential Scanning Calorimetery (DSC) Characterization

DSC analyses can be conducted by using a TA Instruments Q1000 model or Mettler Toledo Model 822e apparatus (or similar). The DSC apparatus is typically calibrated using indium metal as reference for melting point temperature and enthalpy of fusion. The DSC spectra are obtained under nitrogen, using a hermetically-sealed aluminum sample pan. Samples were typically used in the “as is” form without any milling applied.

Method 4M. Hygroscopicity Analysis

The hygroscopicity of salts, solvates and polymorphs of the invention can be assessed by both static and dynamic hygroscopicity studies. For the latter, dynamic vapor sorption/desorption (DVS) experiments can be performed on an SGA-100 gravimetric sorption analyzer (or similar). The experimental protocol typically included full weight equilibrium at 0% RH.

Method 4N: X-Ray Crystallography

In a typical experiment, one single crystal was mounted using a glass fiber on the goniometer. Data were collected on an Enraf-Nonius CAD-4 automatic diffractometer (or similar) using ω/2 theta scans at 293±2 K, unless otherwise noted. The DIFRAC program (Flack, H. D.; Blanc, E.; Schwarzenbach, D. J. Appl. Cryst, 1992, 25, 455-459) was used for centering, indexing and data collection. Two standard reflections were measured every 100 reflections, any observable intensity decay was observed during data collection and is noted for individual structures. The data were corrected for absorption by empirical methods based on psi scans and reduced with the NRCVAX programs (Gabe, E. J.; Le Page, Y.; Charland, J.-P.; Lee, F. L.; White, P. S. J. Appl. Cryst. 1989, 22, 384-387). They were solved using SHELXL-9 and refined by full-matrix least squares on F² with SHELXL-97. (Release 97-2; Sheldrick, G. M. Acta. Cryst. 2008, A64, 112-122.) The non-hydrogen atoms were refined anisotropically. The hydrogen atoms were placed at idealized calculated geometric position and refined isotropically using a riding model. The final absolute structure was assigned by anomalous dispersion effect, unless otherwise noted. (Flack, H. D. Acta Cryst. A 1983, 39, 876-881.)

Table 1 provides an example of how these methods can be used to characterize a representative solvate of the present invention.

TABLE 1
Representative Analytical Tests for Compound 298•HCl H₂O

| Test Parameter | Test Method | Target Results |
|---|---|---|
| Appearance | Method 4A | white to off- |
| | | white powder |
| Identity | | |
| ¹H NMR | Method 4D | conforms to structure |
| ¹³C NMR | Method 4D | conforms to structure |
| ¹⁹F NMR | Method 4D | conforms to structure |
| Purity (HPLC Area %) | Method 4B | >98.5% |
| Total Impurities | Method 4B | ≦1.5% |
| Moisture (Karl Fischer) | Method 4G | ≦5.0% |
| Chloride Ion | Method 4F | 6.0 ± 1.0% |
| Residue on Ignition | Method 4H | <0.2% |
| Endotoxin Levels | Method 4I | ≦10 EU/mg |
| Bioburden | Method 4J | ≦10 CFU/g |

The indicated target results are often modified, typically to more stringent limits, during the progression of an active ingredient through the regulatory process.

Other analytical methods can be employed for the characterization of pharmaceutical compositions of the present invention.

Method 4O. Appearance of Pharmaceutical Composition

Perform visual inspection of the pharmaceutical composition against a clean sheet of white paper and record the observations, including specifically any particulate matter seen.

Method 4P. HPLC Analysis for Identity, Impurities and Potency

A single HPLC method can be used for all three determinations. The same assay can be employed to ascertain stability of the pharmaceutical composition over time at various storage conditions.

### Procedure

Chromatograph standard and sample preparations are injected in such a sequence that standard bracketing is used with every four injections of the sample preparation. Inject each sample preparation in a single injection. The potency, as % free base, can be calculated as shown below. Similarly, the % area of any known and unknown impurities/related substances observed can be calculated as shown below (along with relative retention time of any unknown impurity).

### Chromatographic Conditions

| Column: | XTerra RP18, 3.5 μm, 4.6 × 100 mm | |
|---|---|---|
| Detection (UV): | 225 | nm |
| Column Temperature: | 30° | C. |
| Injection Volume: | 20 | μL |
| Flow Rate: | 1.2 | mL/min |
| Run Time: | 46 | min |
| Data Acquisition Time: | 37.5 | min |

| | |
|---|---|
| Mobile Phase A: | 10 mM Ammonium Hydroxide in Water |
| Mobile Phase B: | 10 mM Ammonium Hydroxide in Acetonitrile |

### Gradient

| Time (min) | % A | % B | Flow (mL/min) |
|---|---|---|---|
| Initial | 80 | 20 | 1.2 |
| 0.5 | 80 | 20 | 1.2 |
| 27 | 60 | 40 | 1.2 |
| 35 | 0 | 100 | 1.2 |
| 37.5 | 0 | 100 | 1.2 |
| 38 | 80 | 20 | 1.2 |
| 46 | 80 | 20 | 1.2 |

### Mobile Phase A Preparation

For each 3 L of mobile phase, pipet 2.25 mL of 25% ammonium hydroxide into 3000 mL of high purity water and mix well.

### Mobile Phase B Preparation

For each three litres of mobile phase, pipet 2.25 mL of 25% ammonium hydroxide into 3000 mL of acetonitrile and mix well.

### Diluent Preparation

For each liter prepared, combine 500 mL high purity water and 500 mL acetonitrile and mix well.

### Standard Preparation

Accurately weigh an amount of the solid reference standard and transfer to a volumetric flask. Dilute to volume with diluent and mix well. Standard preparations are typically stable for at least 7 days when stored at 5° C. or at room temperature unprotected from light.

### Sample Preparation

Dilute the sample with diluent to a previously established working concentration, such as 0.5 mg/mL. Hence, for a 2 mg/mL label claim sample solution, pipet 1.0 mL of sample and 3.0 mL of diluent into a vial and mix well. Prepare sample in duplicate. Each sample is injected once.

### Calculations

The following calculations refer to a pharmaceutical composition formulated at 2 mg/mL.

$$\% of compound = \frac{Sample Peak Area}{Avg STD Peak Area} * \frac{STD Wt ( mg ) \times PF}{50 mL} * \frac{DF}{Label Claim ( mg \text{/} mL )} * 100$$

PF is the potency factor assigned to the pharmaceutical composition. The value is reported in decimal form based on the free base content in the reference standard.
Avg STD Peak Area is the average of all standards throughout the analysis.
DF is the dilution factor applied to a sample. For a 2 mg/mL formulation, the dilution factor is 4 since the sample is diluted 1:4 to reach the final concentration of 0.5 mg/ml.
% of compound is the theoretical concentration reported in mg/mL, typically of free base.
   % Impurity (% area)=The values are calculated by the chromatographic system for each peak relative to the total peak area. Only peaks with area %≧0.05% (LOQ) are integrated.
   % Total Related Substances=sum of Unidentified impurities+sum of Identified impurities
   Note that solvent front, diluent-related peaks are not to be taken into account for the calculation.
   Identification Test: The retention time of the compound in the sample preparation is the same as that in the reference standard preparation (tolerance±5%).

Method 4Q. pH Determination

The pH of the sample can be determined according to USP <791> pH Determination.

Method 4R. Osmolality Determination

The osmolality of the sample can be determined according to USP <785> Osmolality and Osmolarity.

Method 4S. Particulate Matter Assay

Particulate matter can be characterized as directed in USP <788> Particulate Matter in Injections using light obscuration particle count test.

Method 4T. Endotoxin Assay

The samples can be analyzed as directed in USP <85> Bacterial Endotoxins Test using the gel clot method.

Method 4U. Sterility Assay

For sterility, the samples can be tested as directed in USP <71> Sterility Tests.

The use of these methods to characterize representative pharmaceutical compositions of the invention is provided in the Examples.

### 5. Biological Methods

Specific assay methods for the human (GRLN, GHS-R1a), swine and rat ghrelin receptors (U.S. Pat. No. 6,242,199, Intl. Pat. Appl. Nos. WO 97/21730 and 97/22004), as well as the canine ghrelin receptor (U.S. Pat. No. 6,645,726), and their use in generally identifying agonists and antagonists thereof are known.

Appropriate methods for determining the functional and in vivo activity of solvates, salts and polymorphs of the present invention that interact at the human ghrelin receptor (GRLN) are also known. For example, a competitive radioligand binding assay, a fluorescence assay or an Aequorin functional assay can be employed (see U.S. Pat. Nos. 7,452,862; 7,476,653; 7,491,695; and U.S. Patent Appl. Publ. Nos. 2008/051383; 2008/194672). In addition, methods established in the art can be used to determine other parameters important for determining suitability as pharmaceutical agents, such as pharmacokinetics.

The pharmacokinetic behavior of salts, solvates and polymorphs of the invention and their pharmaceutical compositions can be ascertained by methods well known to those skilled in the art and can be used to investigate the pharmacokinetic parameters (elimination half-life, total plasma clearance, etc.) for intravenous, subcutaneous and oral administration of these substances. (Wilkinson, G. R. “Pharmacokinetics: The Dynamics of Drug Absorption, Distribution, and Elimination” in Goodman & Gilman's The Pharmacological Basis of Therapeutics, Tenth Edition, Hardman, J. G.; Limbird, L. E., Eds., McGraw Hill, Columbus, Ohio, 2001, Chapter 1.) See also U.S. Pat. Nos. 7,476,653; 7,491,695; and U.S. Patent Appl. Publ. 2008/0194672. The determination of these parameters for representative pharmaceutical compositions of the invention is presented in the Examples.

### 6. Methods of Use

The salts, solvates and polymorphs of the present invention can be used for the prevention and treatment of a range of medical conditions including, but not limited to, metabolic and/or endocrine disorders, gastrointestinal disorders, cardiovascular disorders, obesity and obesity-associated disorders, central nervous system disorders, bone disorders, genetic disorders, hyperproliferative disorders, disorders characterized by apoptosis, inflammatory disorders and combinations thereof where the disorder may be the result of multiple underlying maladies. In particular embodiments, the disease or disorder is irritable bowel syndrome (IBS), non-ulcer dyspepsia, Crohn's disease, gastroesophageal reflux disorders, gastrointestinal dysmotility occurring in conjunction with other disease states, constipation, ulcerative colitis, pancreatitis, infantile hypertrophic pyloric stenosis, carcinoid syndrome, malabsorption syndrome, atrophic colitis, gastritis, gastric stasis, gastrointestinal dumping syndrome, postgastroenterectomy syndrome, celiac disease, an eating disorder or obesity. In other embodiments, the disease or disorder is congestive heart failure, ischemic heart disease or chronic heart disease. In still other embodiments, the disease or disorder is osteoporosis and/or frailty, accelerating bone fracture repair, metabolic syndrome, attenuating protein catabolic response, cachexia, protein loss, impaired or risk of impaired wound healing, impaired or risk of impaired recovery from burns, impaired or risk of impaired recovery from surgery, impaired or risk of impaired muscle strength, impaired or risk of impaired mobility, altered or risk of altered skin thickness, impaired or risk of impaired metabolic homeostasis or impaired or risk of impaired renal homeostasis. In other embodiments, the disease or disorder involves facilitating neonatal development, stimulating growth hormone release in humans, maintenance of muscle strength and function in humans, reversal or prevention of frailty in humans, prevention of catabolic side effects of glucocorticoids, treatment of osteoporosis, stimulation and increase in muscle mass and muscle strength, stimulation of the immune system, acceleration of wound healing, acceleration of bone fracture repair, treatment of renal failure or insufficiency resulting in growth retardation, treatment of short stature, treatment of obesity and growth retardation, accelerating the recovery and reducing hospitalization of burn patients, treatment of intrauterine growth retardation, treatment of skeletal dysplasia, treatment of hypercortisolism, treatment of Cushing's syndrome, induction of pulsatile growth hormone release, replacement of growth hormone in stressed patients, treatment of osteochondrodysplasias, treatment of Noonans syndrome, treatment of schizophrenia, treatment of depression, treatment of Alzheimer's disease, treatment of emesis, treatment of memory loss, treatment of reproduction disorders, treatment of delayed wound healing, treatment of psychosocial deprivation, treatment of pulmonary dysfunction, treatment of ventilator dependency; attenuation of protein catabolic response, reducing cachexia and protein loss, treatment of hyperinsulinemia, adjuvant treatment for ovulation induction, stimulation of thymic development, prevention of thymic function decline, treatment of immunosuppressed patients, improvement in muscle mobility, maintenance of skin thickness, metabolic homeostasis, renal homeostasis, stimulation of osteoblasts, stimulation of bone remodeling, stimulation of cartilage growth, stimulation of the immune system in companion animals, treatment of disorders of aging in companion animals, growth promotion in livestock, and/or stimulation of wool growth in sheep. Still other embodiments provide for methods of treatment for disorders characterized by apoptosis, such as spinal cord injury and radiation-combined injury. Other embodiments provide for methods of treatment of inflammatory disorders, including ulcerative colitis, inflammatory bowel disease, Crohn's disease, pancreatitis, rheumatoid arthritis, osteoarthritis, asthma, vasculitis, psoriasis, allergic rhinitis, peptic ulcer disease, postoperative intra-abdominal sepsis, ischemia-reperfusion injury, pancreatic and liver damage, sepsis and septic shock, gastric damage caused by certain drugs, stress-induced gastric damage, gastric damage caused by H. pylori, inflammatory pain, chronic kidney disease and intestinal inflammation.

According to a further aspect of the invention, there is provided a method for the treatment of postoperative ileus, gastroparesis, such as that resulting from type I or type II diabetes, other gastrointestinal disorders, cachexia (wasting syndrome), such as that caused by cancer, AIDS, cardiac disease and renal disease, growth hormone deficiency, bone loss, and other age-related disorders in a human or animal patient suffering therefrom, which method comprises administering to said patient an effective amount of at least one member selected from the solvates, salts and polymorphs disclosed herein having the ability to stimulate the ghrelin receptor. Other diseases and disorders treated by the compounds disclosed herein include short bowel syndrome, gastrointestinal dumping syndrome, postgastroenterectomy syndrome, celiac disease, and hyperproliferative disorders such as tumors, cancers, and neoplastic disorders, as well as premalignant and non-neoplastic or non-malignant hyperproliferative disorders. In particular, tumors, cancers, and neoplastic tissue that can be treated by the present invention include, but are not limited to, malignant disorders such as breast cancers, osteosarcomas, angiosarcomas, fibrosarcomas and other sarcomas, leukemias, lymphomas, sinus tumors, ovarian, uretal, bladder, prostate and other genitourinary cancers, colon, esophageal and stomach cancers and other gastrointestinal cancers, lung cancers, myelomas, pancreatic cancers, liver cancers, kidney cancers, endocrine cancers, skin cancers and brain or central and peripheral nervous (CNS) system tumors, malignant or benign, including gliomas and neuroblastomas.

In particular embodiments, the salts, solvates and polymorphs of the present invention can be used to treat postoperative ileus. In other embodiments, salts, solvates and polymorphs of the present invention can be used to treat gastroparesis. In still other embodiments, the solvates, salts and polymorphs of the present invention can be used to treat diabetic gastroparesis or postsurgical gastroparesis. In another embodiment, the solvates, salts and polymorphs of the present invention can be used to treat opioid-induced bowel dysfunction.

In particular embodiments, salts, solvates and polymorphs of the present invention can be used to treat postoperative ileus, gastroparesis, diabetic gastroparesis, postsurgical gastroparesis, opioid-induced bowel dysfunction, chronic intestinal pseudo-obstruction, acute colonic pseudo-obstruction (Ogilvie's syndrome), short bowel syndrome, emesis, constipation-predominant irritable bowel syndrome (IBS), chronic constipation, functional dyspepsia, cancer-associated dyspepsia syndrome, graft versus host disease, gastroesophageal reflux disease (GERD), gastric ulcers, Crohn's disease, gastroenteritis, gastrointestinal dysfunction or delayed gastric emptying in patients with eating disorders, including anorexia nervosa and bulimia, gastrointestinal dysfunction or delayed gastric emptying in patients with Parkinson's disease, gastrointestinal dysfunction or delayed gastric emptying in patients with myotonic muscular dystrophy, gastrointestinal dysfunction or delayed gastric emptying in patients with autonomic degeneration, gastrointestinal dysfunction or delayed gastric emptying in patients who have suffered a stroke, gastrointestinal dysfunction or delayed gastric emptying in patients with multiple sclerosis, gastrointestinal dysfunction or delayed gastric emptying in patients with neurological diseases and disorders, including amyloid neuropathy, primary dysautonomia, vagal injury and pyloric stenosis, gastrointestinal dysfunction or delayed gastric emptying in patients with psychiatric diseases, including depression, gastrointestinal dysfunction or delayed gastric emptying in patients with scleroderma, gastrointestinal dysfunction or delayed gastric emptying in patients with cystic fibrosis, gastrointestinal dysfunction or delayed gastric emptying in patients with connective tissue diseases, including, systemic sclerosis, dermatomyositis, polymyositis, systemic lupus erythematosis and amyloidosis, gastrointestinal dysfunction or delayed gastric emptying in patients with liver cirrhosis, gastrointestinal dysfunction or delayed gastric emptying in patients with liver failure, gastrointestinal dysfunction or delayed gastric emptying in patients with renal failure, gastrointestinal dysfunction or delayed gastric emptying in patients with gallbladder disorders, gastrointestinal dysfunction or delayed gastric emptying in patients with migraines, gastrointestinal dysfunction or delayed gastric emptying with sepsis, gastrointestinal dysfunction or delayed gastric emptying in patients with brain stem lesions, gastrointestinal dysfunction or delayed gastric emptying in patients with spinal cord injury, gastrointestinal dysfunction or delayed gastric emptying in patients with cancer, including stomach, biliary, esophageal, gastric and pancreatic cancers, gastrointestinal dysfunction or delayed gastric emptying in patients with neoplasia, gastrointestinal dysfunction or delayed gastric emptying in patients who have undergone radiation treatment, gastrointestinal dysfunction or delayed gastric emptying in patients with achalasia, gastrointestinal dysfunction or delayed gastric emptying in patients with infectious diseases, including HIV, Herpes zoster infection and Chagas disease, gastrointestinal dysfunction or delayed gastric emptying as a result of surgery, gastrointestinal dysfunction or delayed gastric emptying in patients with critical illness, gastrointestinal dysfunction or delayed gastric emptying in patients requiring critical care, gastrointestinal dysfunction or delayed gastric emptying in patients after transplants, including heart or lung transplantation, gastrointestinal dysfunction or delayed gastric emptying in patients with Turner's syndrome, gastrointestinal dysfunction or delayed gastric emptying as a result of treatment with pharmaceutical agents, including opioids, anticholinergics, beta blockers, calcium channel antagonists, glucagon-like peptide-1 (GLP-1) receptor agonists, amylin receptor agonists, peptide YY (PYY) receptor agonists, proteasome inhibitors, tricyclic antidepressants, monoamine uptake blocker antidepressants, cancer chemotherapy agents, adrenergic agonists, dopaminergic agents, antimalarials, antispasmodics, cannabinoid agonists, octreotide, levodopa, alcohol and nicotine, gastrointestinal dysfunction or delayed gastric emptying as a result of endocrine disturbances, including hypothyroidism, hypethyroidism, Addison's disease and porphyria, gastrointestinal dysfunction or delayed gastric emptying as a result of metabolic disturbances, including hyperglycemia, hypokalemia and hypomagnesemia, gastrointestinal dysfunction or delayed gastric emptying as a result of anesthesia, gastrointestinal dysfunction or delayed gastric emptying as a result of mechanical ventilation, gastrointestinal dysfunction or delayed gastric emptying as a result of electrolyte disturbances, gastrointestinal dysfunction or delayed gastric emptying as a result of severe trauma or gastrointestinal dysfunction or delayed gastric emptying as a result of pain.

The present invention further provides methods of treating a horse or canine for a gastrointestinal disorder comprising administering a therapeutically effective amount of a salt, solvate or polymorph of the invention. In some embodiments, the gastrointestinal disorder is ileus or colic.

As used herein, “treatment” is not necessarily meant to imply cure or complete abolition of the disorder or symptoms associated therewith.

The salts, solvates or salts of the present invention can further be utilized for the preparation of a pharmaceutical composition or medicament for the treatment of a range of medical conditions including, but not limited to gastrointestinal disorders, metabolic and/or endocrine disorders, cardiovascular disorders, central nervous system disorders, obesity and obesity-associated disorders, genetic disorders, bone disorders, hyperproliferative disorders, disorders characterized by apoptosis and inflammatory disorders.

Further embodiments of the present invention will now be described with reference to the following examples. It should be appreciated that these examples are for the purposes of illustrating embodiments of the present invention, and do not limit the scope of the invention.

### EXAMPLES

### Example 1

### Preparations of Compound 298.HCl.H₂O by Crystallization

### Preparation A

Amorphous compound 298.HCl (1.0 g) was suspended in hot H₂O and methylethylketone (MEK) (4:1) added dropwise until complete dissolution. The solution was then slowly cooled to room temperature using an oil bath (90° C.->25° C.) before being placed at 4° C. overnight (O/N). The resulting crystals of compound 298.HCl.H₂O were collected by filtration and dried O/N in air. Yield: 82%.

### Preparation B

Amorphous compound 298.HCl (3.0 g) was dissolved in 40 mL of hot H₂O/MEK (3:1). The solution was slowly cooled to room temperature using an oil bath (90° C.->25° C.), then placed at 4° C., O/N. The resulting crystals were filtered, then dried 24 h in air. Same unit cell was obtained for the X-ray structure as for the crystals of compound 298.HCl.H₂O formed in Preparation A.

Similar results were obtained by conducting the crystallization from 40 mL of hot H₂O/iPrOH (3:1).

### Preparation C

A suspension of compound 298.HCl.EtOH (10 g, 16.1 mmol) in H₂O/MEK (8:2, 100 mL) was stirred at reflux and MEK slowly added until complete dissolution. The mixture was slowly cooled to room temperature, then left at RT for 10 h. The crystals were collected by filtration and washed with cold water (1×10 mL). The solid was dried overnight (16-18 h) in air to give compound 298.HCl.H₂O as large white crystals (˜80% yield).

### Preparation D

To a solution of compound 298 free base (100 mg, 0.18 mmol, 1.0 eq) in MEK (0.5 mL) was slowly added concentrated HCl (23 μL, 0.27 mmol, 1.5 eq) in an 8 mL glass vial. The solution was stirred 10 min at RT, during which time precipitation occurs, then 0.5 mL of water added, which results in complete dissolution of the precipitate. The solution was concentrated to 0.5 mL under a nitrogen atmosphere and 0.5 mL of water added. Precipitation occurs. The suspension was heated to reflux and MEK added dropwise until complete dissolution. The solution was slowly cooled to RT in an oil bath (90° C.->RT). Crystallization occurred upon cooling. The vial was placed at approximately 5° C., O/N. The crystals were collected by filtration and washed with cold water (1×0.5 mL). The crystals were dried under high vacuum at 50° C. which provided compound 298.HCl.H₂O as white crystals (70 mg, 70%).

### Preparation E

To a solution of compound 298 (38.0 g, 70.6 mmol) in 115 mL absolute EtOH was added 1.25 M HCl in EtOH (113 mL, 141 mmol, Fluka), The mixture was stirred for 15 min at RT, then 30 min at 0° C. The precipitated solid was collected by filtration while still cold, then washed with cold EtOH (2×100 mL). The solid is dried in vacuo O/N to give 26.1 g (64%) of compound 298.HCl.EtOH as a white solid. This was dissolved in 210 mL EtOH/H₂O (85:15) and heated to 75° C. The solution was permitted to cool to RT, then placed at −20° C., O/N. The crystals that formed were collected, washed with abs. EtOH (1×100 mL), then dried in vacuo to provide 25.7 g (99%) of white crystalline solid. This was combined with 3.3 g of identical material (by HPLC and MS) prepared similarly and the combined solid dissolved in EtOH/H₂O (3:1, 125 mL), heated to 75° C., filtered while still hot, and the filtrate cooled to RT, then placed at −20° C., O/N. The crystallized material was collected, washed with EtOH (1×) and dried in vacuo to leave 24.4 g. To this was added iPrOH/H₂O (7:3, 180 mL) and the mixture heated to 85° C. until dissolution. The solution was permitted to cool to RT, then placed at −20° C. A gummy solid separated, from which the solvent was decanted and the residue treated with acetone. The solid that formed was collected, rinsed with acetone and dried in vacuo to yield 19.0 g. This material in turn was dissolved in MEK/H₂O (15:85, 147 mL) and heated to 95° C. Upon dissolution, the heating was stopped, the solution allowed to cool to RT, then placed at 2° C., O/N. The solid was collected, washed with cold H₂O (2×) and dried in vacuo to provide 14.5 g of crystalline material. Most (13.0 g) of this solid was suspended in 73 mL H₂O and heated to 95° C. (oil bath), then MEK added dropwise until complete dissolution (16 mL). The solution was slowly (5° C./hr) cooled to RT and left O/N. The precipitated solid was collected and washed with H₂O (2×) to leave 11.6 g of white crystals. This solid was dissolved in MEK/H₂O (1:4, 65 mL) at 95° C. (oil bath). The solution was slowly (5° C./hr) cooled to RT. The crystals that formed were collected, washed with MEK/H₂O (1:4, 2×25 mL), H₂O (1×25 mL), then dried in air O/N to provide compound 298.HCl.H₂O (7.6 g) as a white crystalline solid.

Purity

The purity of the solvate was determined by HPLC using Method 4B and UV detection at 230 nm.

X-Ray Crystallography

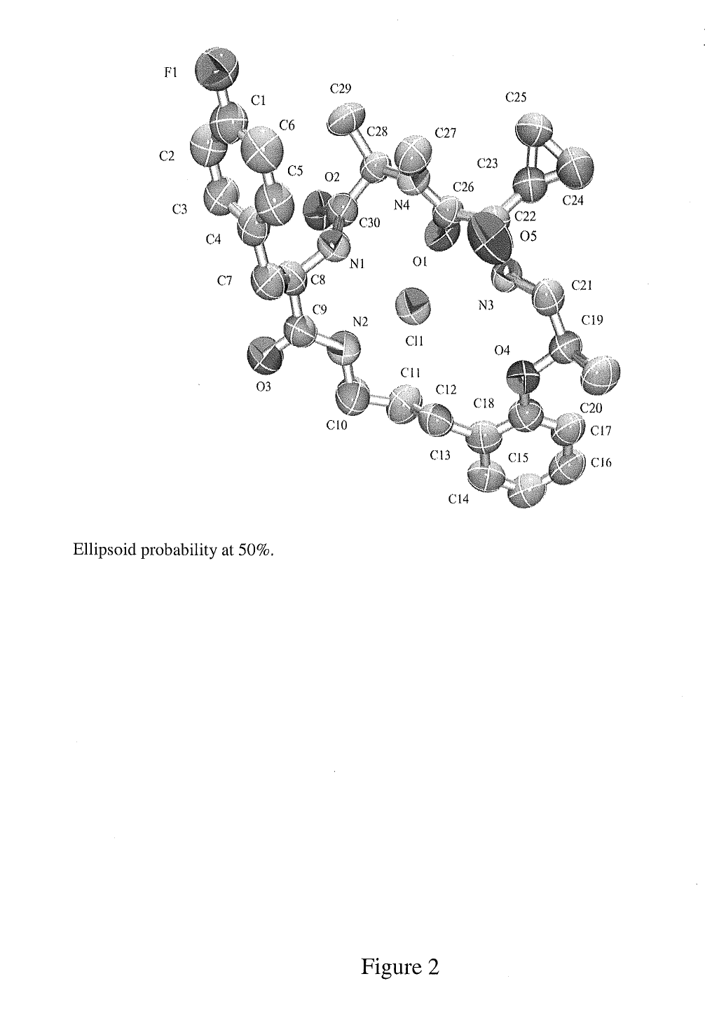

A representative X-ray crystal structure of compound 298.HCl.H₂O using Method 4N is presented in FIG. 2. This confirmed the monohydrochloride monohydrate structure and was consistent with the X-ray structures for the solvate formed by other methods.

### Crystal Data and Structure Refinement for Compound 298.HCl.H₂O

| Empirical formula | C₃₀H₄₂ClFN₄O₅ | |
|---|---|---|
| Formula weight | 593.13 | |
| Temperature | 293(2) K | |
| Wavelength | 1.54176 Å | |
| Crystal system | Orthorhombic | |
| Space group | p212121 | |
| Unit cell dimensions | a = 10.535(2) Å | α = 90°. |
| | b = 13.352(9) Å | β = 90°. |
| | c = 22.368(6) Å | γ = 90°. |
| Volume | 3146(2) Å³ | |
| Z | 4 | |
| Density (calculated) | 1.252 Mg/m³ | |
| Absorption coefficient | 1.484 mm⁻¹ | |
| F(000) | 1264 | |
| Crystal size | 0.60 × 0.40 × 0.30 mm³ | |
| Theta range for data collection | 3.86 to 69.92°. | |
| Index ranges | 0 <= h <= 12, 0 <= k <= 16, | |
| | 0 <= 1 <= 27 | |
| Reflections collected | 3264 | |
| Independent reflections | 3264 [R(int) = 0.0000] | |
| Completeness to theta = | 97.0% | |
| 69.92° | | |
| Absorption correction | Empirical | |
| Max. and min. transmission | 0.6644 and 0.4696 | |
| Refinement method | Full-matrix least-squares | |
| | on F² | |
| Data/restraints/parameters | 3264/2/383 | |
| Goodness-of-fit on F² | 0.948 | |
| Final R indices [I > | R1 = 0.0661, wR2 = 0.1649 | |
| 2sigma(I)] | | |
| R indices (all data) | R1 = 0.1081, wR2 = 0.1929 | |
| Absolute structure parameter | 0.13(4) | |
| Extinction coefficient | 0.0100(10) | |
| Largest diff. peak and hole | 0.249 and −0.223 e.Å⁻³ | |
| Intensity decay during | None | |
| data collection | | |
| Final BASF parameter | 0.13 | |

Solubility

The solubility of compound 298HCl.H₂O was determined in aqueous (pH 4.0-7.0) and non aqueous media. The data from these studies are summarized in Table 2.

TABLE 2
Solubility Data of Compound 298•HCl•H₂O

| Solvent | Solubility |
|---|---|
| Water, pH 4.0 | 7 mg/mL |
| Water, pH 5.0 | 7 mg/mL |
| Water, pH 6.0 | 2 mg/mL |
| Water, pH 7.0 | 0.2 mg/mL |
| 0.9% Saline, pH 6.0 | 1 mg/mL |
| 5% Dextrose, 10 mM acetate | 8 mg/mL |
| buffer, pH 4.5 | |
| Methanol | Soluble¹ |
| Dimethylsulfoxide | Sparingly soluble¹ |
| 2-Butanol | Slightly soluble¹ |
| Ethanol | Slightly soluble¹ |
| Acetonitrile | Slightly soluble¹ |
| Tetrahydrofuran | Slightly soluble¹ |
| Isopropanol | Very slightly soluble¹ |
| Ethyl Acetate | Very slightly soluble¹ |

¹Solubility descriptions follow the guidance from USP vol. 28 (2005), p. 9 (General Notices section).

UV Analysis

The ultraviolet spectrum was obtained using Method 4C for a solution of 0.1 mg/mL of compound 298.HCl.H₂O in MeOH. Under these conditions, the solvate exhibited maxima at 217, 266, 272, and 278 nm.

NMR Analysis

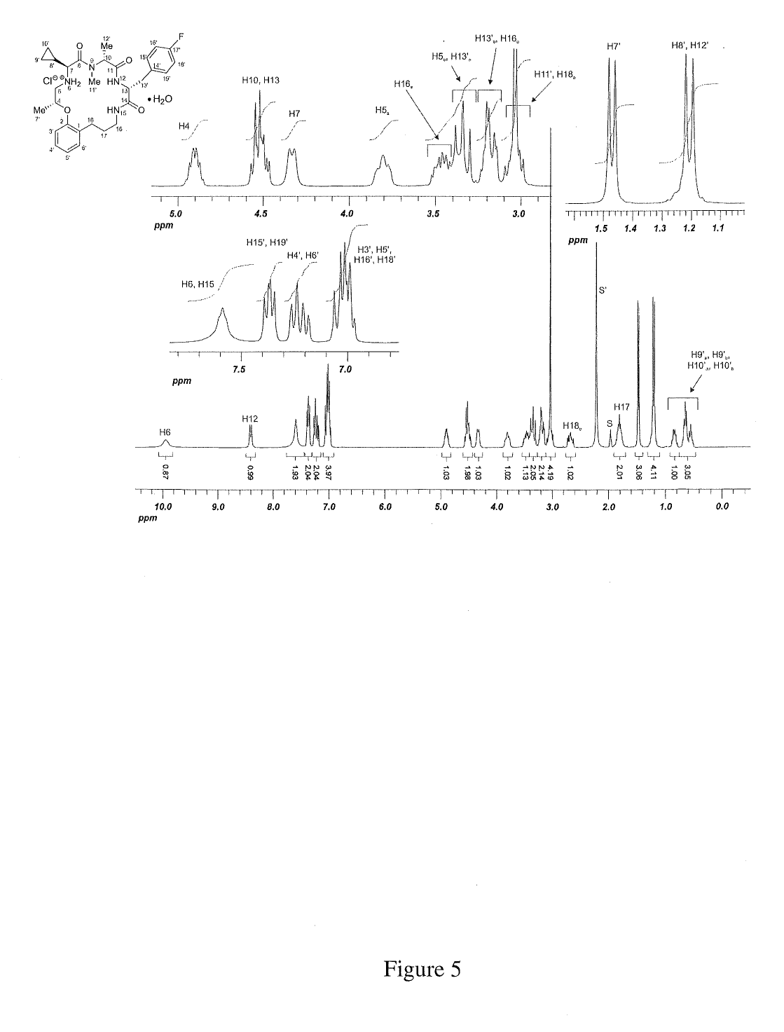

The ¹H, ¹³C and ¹⁹F NMR spectra of compound 298.HCl.H₂O were obtained using Method 4D with a Varian Mercury-VX 300 MHz. Specifically, the ¹H NMR (1D and 2D) spectra were obtained with the spectrometer operating at 300.080 MHz and maintained at 25° C. The sample was prepared by dissolving 36.1 mg of compound 298.HCl.H₂O in 3.61 mL of CD₃CN (99.96% D, Aldrich). The chemical shift reference in all ¹H NMR spectra was provided by the CHD₂CN quintet (δ=1.94 ppm). A representative ¹H NMR spectrum is displayed in FIG. 5. These spectral data fully conform to the structure of compound 298.HCl.H₂O.

¹H NMR (CD₃CN): δ 0.48-0.70 (m, 3H), 0.77-0.89 (m, 1H), 1.23 (d, m, overlapping, J=7.5 Hz, 4H), 1.47 (d, J=6.1 Hz, 3H), 1.70-1.90 (m, 2H), 2.58-2.78 (m, 1H), 2.96-3.12 (s, m, overlapping, 4H), 3.12-3.26 (m, 2H), 3.26-3.40 (m, 2H), 3.40-3.55 (m, 1H), 3.72-3.88 (m, 1H), 4.33 (d, J=8.3 Hz, 1H), 4.46-4.60 (m, 2H), 4.85-4.97 (m, 1H), 6.93-7.08 (m, 4H), 7.16-7.30 (m, 2H), 7.32-7.43 (m, 2H), 7.51-7.67 (br s, br t, overlapping, 2H), 8.38 (d, J=9.3 Hz, 1H), 9.80-10.05 (br s, 1H).

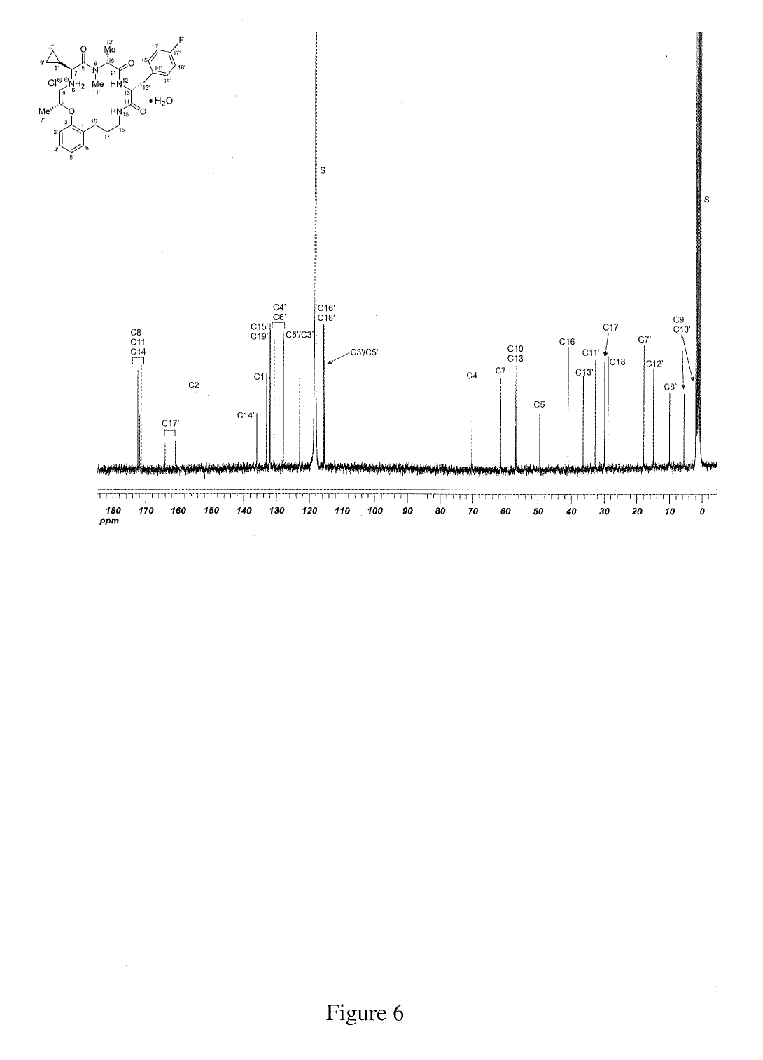

The ¹³C NMR spectrum was obtained on the spectrometer operating at 75.46 MHz and maintained at 25° C. The same sample that had been prepared for the ¹H NMR experiments was used for ¹³C NMR spectroscopy. The chemical shift reference was provided by the CD₃CN septet ( [symbol] =1.32 ppm). FIG. 6 shows a representative ¹³C NMR spectrum of compound 298.HCl.H₂O.

¹³C NMR (CD₃CN): [symbol] 1.9, 5.6, 10.1, 15.1, 18.1, 28.9, 30.0, 32.8, 36.4, 41.1, 49.5, 56.7, 57.0, 61.5, 70.3, 115.3, 115.6 (J_{C-F}=21 Hz), 123.0, 127.9, 130.8, 132.0 (J_{C-F}=8 Hz), 133.1, 136.0 (J_{C-F}=3 Hz), 154.9, 162.4 (J_{C-F}=242 Hz), 171.4, 171.8, 172.4.

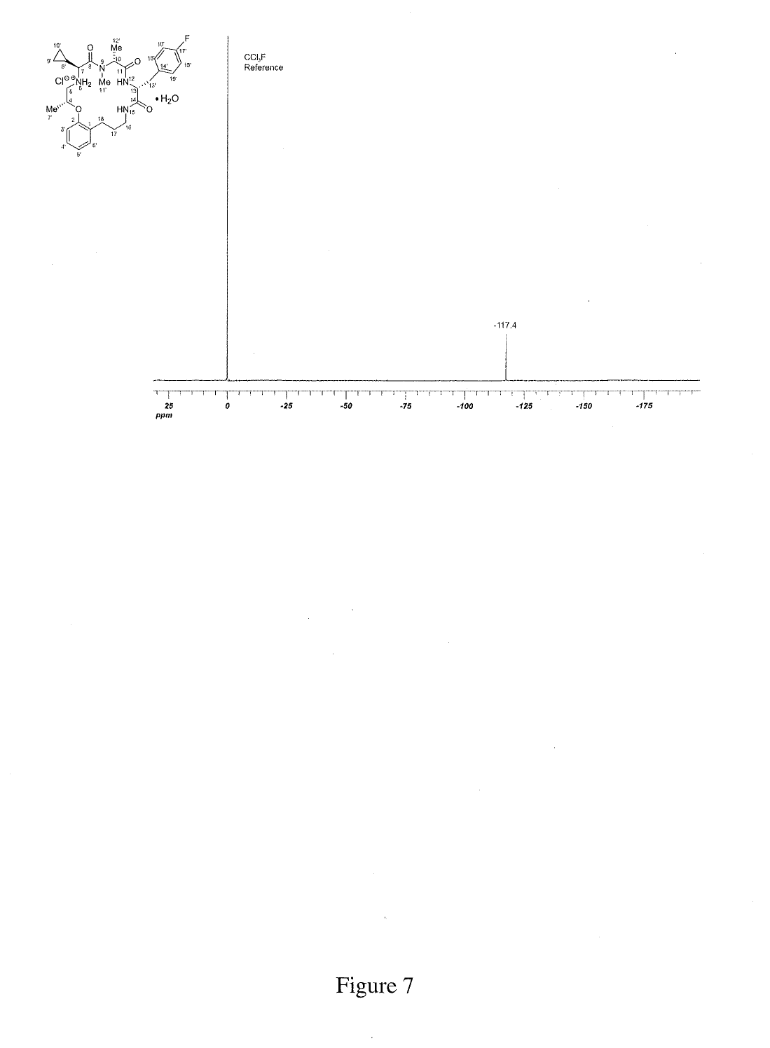

Lastly, the ¹⁹F NMR spectrum was obtained on the spectrometer operating at 282.33 MHz and maintained at 25° C. The sample was prepared just as was done for ¹H NMR spectroscopy except for the addition of a drop of CCl₃F used as the reference standard (δ=0 ppm). As shown in the representative spectrum in FIG. 7, a single ¹⁹F signal at −117.4 ppm was obtained as expected for the structure.

FT-IR Analysis

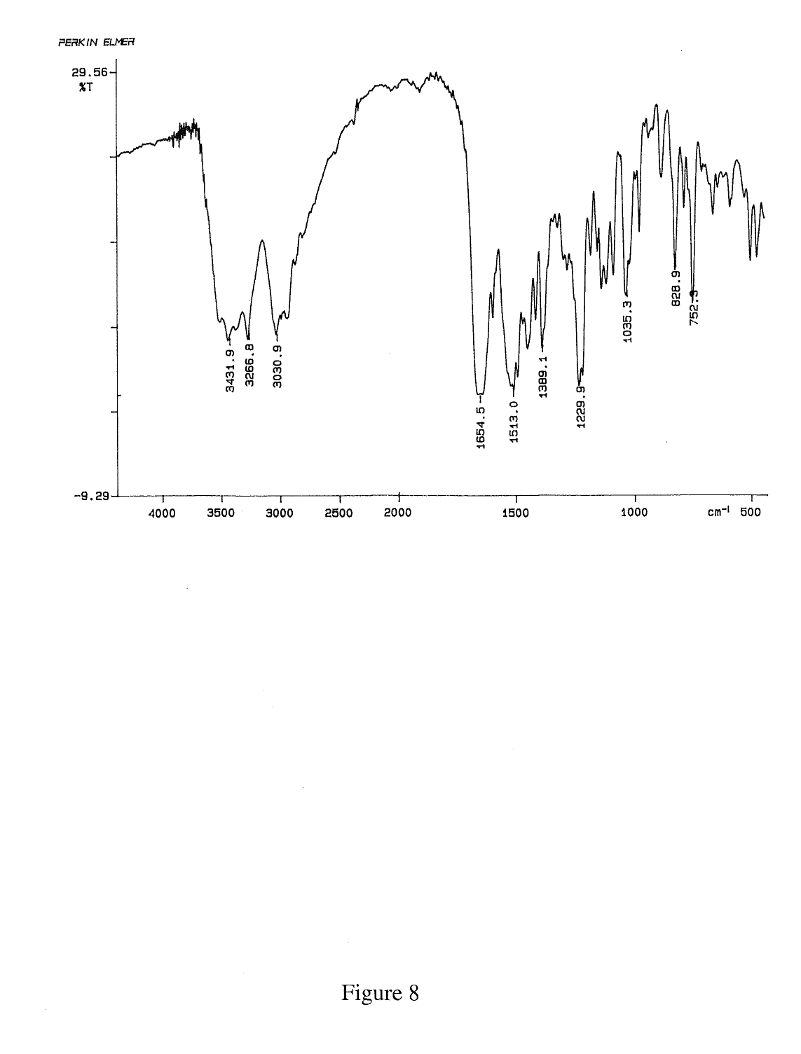

The Fourier transformed infrared (FT-IR) absorption spectrum of compound 298.HCl.H₂O was obtained as a potassium bromide pellet using Method 4E. The spectrum was obtained by averaging 16 scans measured with a resolution of 4 cm⁻¹. The FT-IR spectrum conforms to the structure with the most prominent bands as assigned in Table 3 and a representative spectrum is furnished in FIG. 8.

TABLE 3
Prominent FT-IR Absorption Bands for Compound 298•HCl•H₂O

| Infrared Absorption Bands | |
|---|---|
| (wavenumber in cm⁻¹) | Infrared Band Assignments¹ |
| 3140-3600 | amide ν_{N—H}² |
| 2700-3140 | secondary ammonium ν_{N—H} |
| | saturated C—H νₛ & νₐₛ |
| 1654 | amide ν_{C═O} (amide I band) |
| 1513 | amide δ_{N—H} (amide II band) |
| 1389 | amide ν_{C—N} (amide III band) |
| 1200-1265 | C—F νₛ; arylalkyl ether C—O νₐₛ |
| 1035 | arylalkyl ether C—O νₛ |
| 829 | para-substituted aromatic ring δ_{C—H} |
| | (out-of-plane) |
| 753 | ortho-substituted aromatic ring δ_{C—H} |
| | (out-of-plane) |

¹Abbreviations used in this Table: ν = stretch vibrational mode. νₛ = symmetric vibrational mode. νₐₛ = anti-symmetric vibrational mode. δ = bend or deformation vibrational mode.

XRPD Analysis

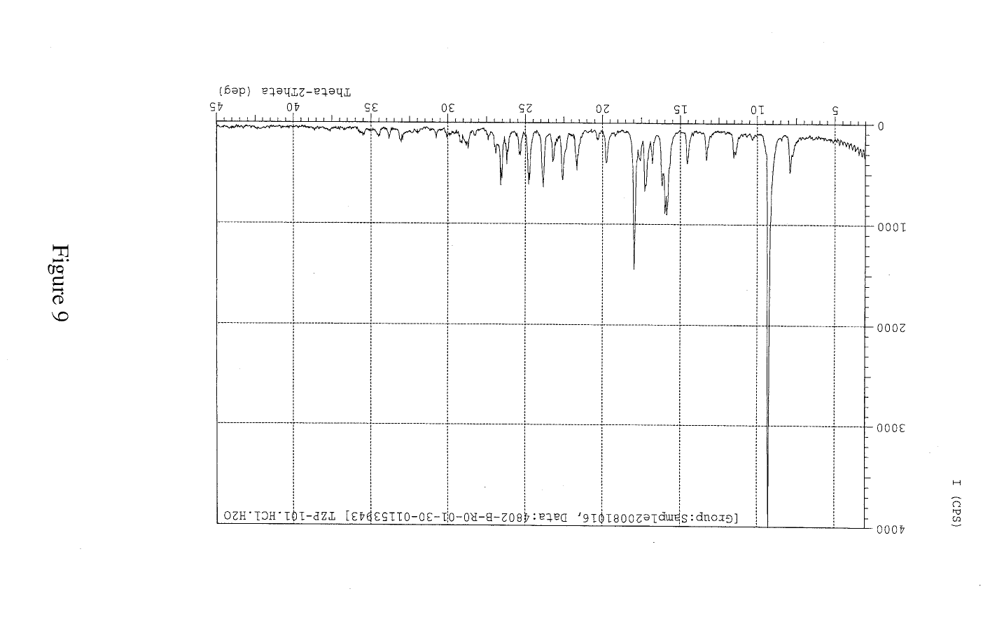

The X-ray powder diffraction (XRPD) analysis for compound 298.HCl.H₂O was conducted according to Method 4K. A representative diffractogram is shown in FIG. 9. Peaks (2θ) were obtained at: 7.7, 7.9, 8.9, 9.3, 9.5, 11.4, 11.5, 13.3, 14.5, 15.6, 15.9, 16.2, 16.8, 17.2, 17.6, 17.9, 19.7, 21.6, 22.3, 22.6, 23.2, 23.9, 24.8, 25.3, 26.2, 26.6, 26.9, 28.6, 29.1, 33.0, 33.8.

DSC Spectrum

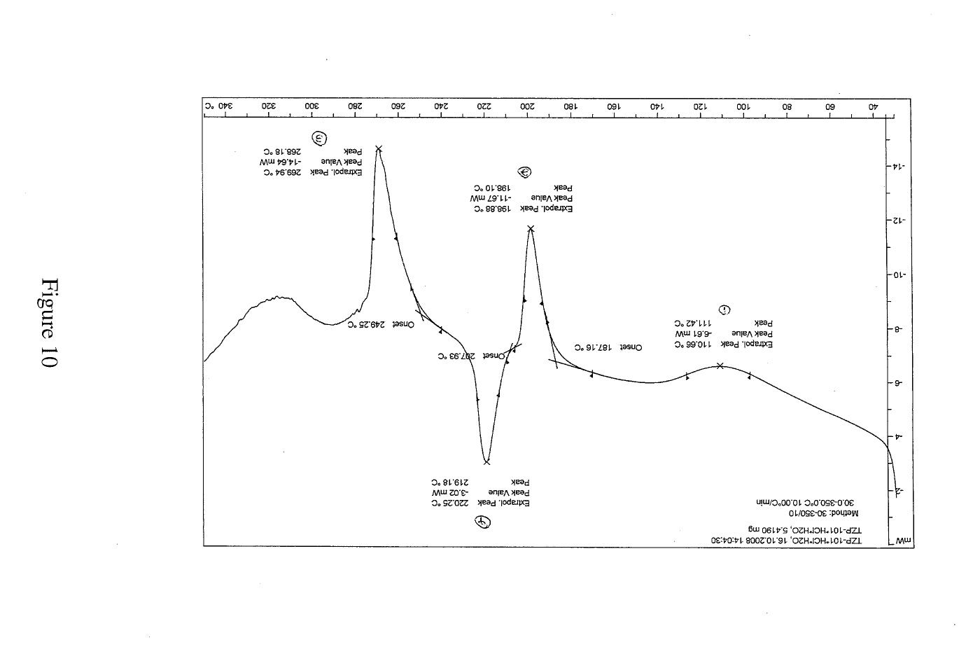

The DSC spectrum for compound 298.HCl.H₂O was obtained using Method 4L and a representative example is provided in FIG. 10.

Hygroscopicity Analysis

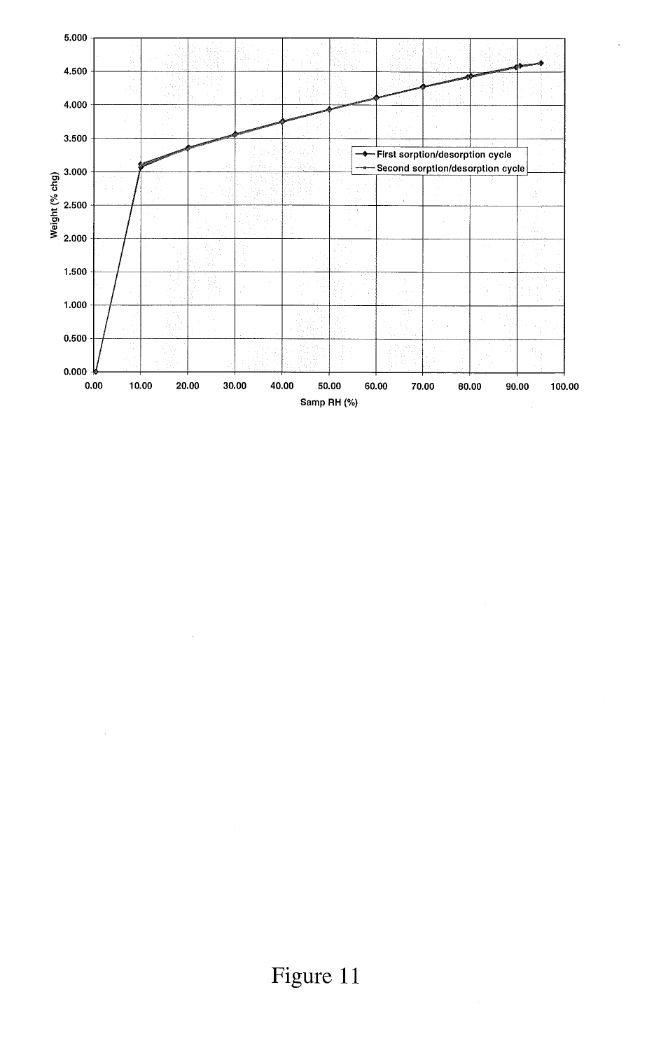

The hygroscopicity of compound 298.HCl.H₂O was assessed by both static and dynamic vapor sorption/desorption (DVS) experiments were performed at 25° C. according to Method 4M. A representative hygroscopicity profile thus assessed through these DVS studies is presented in FIG. 11.

Only a 1% weight change (ca. 0.3 H₂O) was observed in the 20%-90% relative humidity range. The lattice water lost through equilibration at 0% RH (2.6% weight loss≡0.9 H₂O) was rapidly regained within the initial 10% RH increase, thus reconstituting the monohydrate form (3% weight H₂O content). Based on these results, compound 298.HCl.H₂O is essentially non-hygroscopic. Additionally, no residual amorphous content was detectable in the bulk crystalline material by this criterion.

The static hygroscopicity study was performed by exposing compound 298.HCl.H₂O for a period of 3 months to 80% RH humidity at 25° C. No deliquescence was detected at any point during this study. The sample was analyzed at 1-week, 1-month, 2-month and 3-month time points by the following methods: DSC, thermogravimetric analysis and Karl-Fischer titrimetry. These studies also confirmed that compound 298.HCl.H₂O is essentially non-hygroscopic.

### Example 2

### Synthesis of Compound 298.HCl.H₂O

### Step 2-1: Preparation of Compound 298.HCl.EtOH

In a 25 L reactor, compound 298 (1.75 kg), prepared as shown in FIG. 1, was dissolved in ethanol (10.5 kg), then 1.5 eq of hydrogen chloride (3.5 L, 1.0 M in ethanol) was added to form the hydrochloride salt. The mixture was stirred at ambient temperature to allow for precipitation of the hydrochloride, then at 0° C. for 30 min. The solid thus obtained was filtered and washed with ethanol, then dried under vacuum at 40° C. The material (1.3 kg, 83% yield, 98.5% HPLC purity) was used directly for the next step.

### Step 2-2: Recrystallization of Compound 298.HCl.EtOH

In a 25 L reactor, compound 298.HCl EtOH (1.3 kg, 2.09 mol) was suspended in a mixture of ethanol (8.5 L) and water (1.5 L). The mixture was brought to reflux until dissolution and filtered hot through a 0.20 μm filter. Cooling at −20° C. provided crystalline compound 298.HCl.EtOH (1.1 kg, 84.6% yield, 99.2% HPLC purity), which was used as such for the next step.

### Step 2-3: Formation and Crystallization of Compound 298.HCl H₂O

In a 25 L reactor, compound 298.HCl.EtOH (1.1 kg, 1.77 mol) was suspended in a mixture of 2-butanone (1.1 L) and water (4.4 L). The mixture was brought to reflux until complete dissolution occurred. It was then cooled to room temperature and maintained at that temperature for 16 h to allow for complete crystallization. The product was isolated by filtration, then washed with cold water to give compound 298.HCl.H₂O (868.5 g, 82.7% yield, 99.6% HPLC purity) as white crystals.

TABLE 4
Analysis of Representative Preparations
of Compound 298•HCl•H₂O

| Test Parameter¹ | Batch 1 | Batch 2 |
|---|---|---|
| Amount Prepared | 0.06 kg | 0.87 kg |
| Appearance | white powder | white powder |
| Identity | | |
| ¹H NMR (DMSO-d₆) | conforms to structure | conforms to structure |
| ¹³C NMR (DMSO-d₆) | conforms to structure | conforms to structure |
| ¹⁹F NMR (DMSO-d₆) | conforms to structure | conforms to structure |
| Purity (HPLC Area %) | 99.30% | 99.6% |
| Total impurities | 0.71% | 0.42% |
| Moisture (Karl Fischer) | 4.40% | 3.9% |
| Chloride ion | 5.88% | 5.9% |
| Residue on ignition | n/a | 0.15% |
| Endotoxin levels | NT² | <0.05 EU/mg |
| Bioburden: | NT² | <0.05 EU/mg |

¹Based upon test methods and target values in Table 1.
²Not tested

### Stability Testing

TABLE 5
Stability of a Representative Preparation of Compound 298•HCl•H₂O (Batch 1)

| | Condition 1: | | Condition 2: | | Condition 3: | |
|---|---|---|---|---|---|---|
| | 5° C. | | 25° C., 60% RH¹ | | 40° C., 75% RH | |
| Time | HPLC Assay | Total | HPLC Assay | Total | HPLC Assay | Total |
| (mos) | (% Purity²) | Impurities (%) | (% Purity²) | Impurities (%) | (% Purity²) | Impurities (%) |
| 0 | 100.48 | 0.41 | 100.48 | 0.41 | 100.48 | 0.41 |
| 1 | NT³ | NT | 98.99 | 0.40 | 98.49 | 0.39 |
| 3 | 98.72 | 0.39 | 98.39 | 0.40 | 98.22 | 0.40 |
| 6 | 98.48 | 0.42 | 98.81 | 0.42 | 98.41 | 0.44 |
| 9 | 100.78 | 0.47 | 100.65 | 0.46 | | |
| 12 | 98.19 | 0.42 | 97.59 | 0.49 | | |
| 18 | 98.68 | 0.43 | 99.81 | 0.21 | | |
| 24 | 100.15 | 0.43 | 100.24 | 0.48 | | |

¹Relative humidity
²By weight using Method 4B
³Not tested

### Example 3

### Preparation and Purification of 298.HCl.H₂O

### Step 3-1: Synthesis of Compound 298.HCl.EtOH

A 100-L glass jacketed reactor under nitrogen was charged with 85.8 L of THF, 4.2 L of diisopropylethylamine (DIPEA) and 1.6 kg of DEPBT. The reactor temperature was set to 20° C., and compound 298 in THF added over 6 h. The solution was stirred at this temperature for at least 36 h after the addition. Upon completion (starting material is <1% by HPLC Area %), the reactor temperature was adjusted to 40° C. and the THF concentrated under vacuum until approximately 20 L of solution remained. 1 M Sodium carbonate (20.7 L), followed by 29.7 L of EtOAc were charged into the reactor, then agitated vigorously for 2 h. The reactor agitation was stopped, and the bottom aqueous layer discharged from the reactor. The organic phase was washed sequentially with 5.3 L of 1 M sodium carbonate during 30 min, then 8 L of saturated aq. sodium chloride. The reactor temperature was adjusted to 40° C. and the organic solution containing EtOAc concentrated under vacuum until approximately 22 L of solution remained. EtOH (44 L) was charged in the reactor and the content of the reactor evaporated at 40° C. until approximately 33 L of solution remained. Ethanol was added to bring the final volume to 44 L. The reactor temperature was adjusted to 15-20° C., and 3.4 L of approximately 2.5 M hydrogen chloride in ethanol added to make the pH 1.76 (target range pH 1.5-2.0). After cooling to 0°±5° C., precipitation occurred overnight. The solid was collected by filtration and dried in vacuo at 25° C. to give 2.2 kg (67.6% yield) of isolated compound 298.HCl.EtOH in 98.8% purity (HPLC Area %).

### Step 3-2: Recrystallization of Compound 298.HCl EtOH

A 100-L glass jacketed reactor was charged with 19.5 L of aqueous ethanol (EtOH/H₂O 85:15, using water for injection), and 2.2 kg of compound 298.HCl.EtOH. The reactor was heated to 75°-85° C., and the solution transferred hot via a transfer line fitted with a 0.2 μm filter (Whatman #6715-7502). The reactor was cleaned with EtOH, and the filtered solution returned to the reactor. The reactor temperature was then adjusted to 20° C. and the content agitated at this temperature for 6 h. The reactor was further cooled to −15° C.±5° C., and the resulting slurry stirred for 2 h. The solid was collected by filtration, washed with ethanol (chilled to −13.9° C.) and dried in vacuo at 25° C. to provide 1.843 kg (84% yield) of crystalline compound 298.HCl.EtOH in 99.7% purity (HPLC Area %).

### Step 3-3: Crystallization of Compound 298.HCl H₂O

A 22-L glass jacketed reactor was charged with 9.2 L of aqueous 2-butanone (MEK/Water for Injection, 1:4), and 1.843 kg of compound 298.HCl.EtOH. The reactor was heated to 75°-85° C. After slow cooling and stirring at 20±5° C., solid compound 298.HCl.H₂O was collected by filtration, washed with water for injection (pre-chilled to 4° C.) and dried under nitrogen at 25° C. This yielded 1.483 kg (84%. yield) of crystalline compound 298.HCl.H₂O (99.9% HPLC purity).

¹³C NMR (DMSO-d₆): δ 1.18, 4.55, 9.53, 14.58, 17.50, 27.54, 28.73, 31.69, 35.81, 48.75, 54.18, 55.69, 59.83, 69.38, 112.81, 114.65 (J_{C-F} 50.1 Hz), 120.98, 126.76, 129.44, 130.94 (J_{C-F} 19.8 Hz), 134.46 (J_{C-F} 7.2 Hz), 154.51, 160.88 (J_{C-F} 577 Hz), 169.66, 170.37, 171.12.

TABLE 6
Representative Physicochemical Characteristics
of Compound 298•HCl•H₂O

| | |
|---|---|
| Appearance | White to off-white powder |
| Solubility | Soluble at 7 mg/mL in water |
| | Soluble at 8 mg/mL in 5% Dextrose, 10 mM acetate |
| | buffer, pH 4.5 |
| | Slightly soluble in ethanol and acetonitrile |
| | Very slightly soluble in ethyl acetate |
| Melting Point | Transition at 197.55° C. by DSC (see FIG. 10) |
| Hygroscopicity | Non-hygroscopic |

TABLE 7
Analysis of Representative Preparations
of Compound 298•HCl H₂O

| Test Parameter¹ | Batch 3 | Batch 4 | Batch 5 |
|---|---|---|---|
| Amount Prepared | 0.025 kg | 0.10 kg | 1.5 kg |
| Appearance | white powder | white powder | white powder |
| Identity | | | |
| ¹H NMR (DMSO-d₆) | conforms to | conforms to | conforms to |
| | structure | structure | structure |
| ¹³C NMR (DMSO-d₆) | NT² | NT² | conforms to |
| | | | structure |
| ¹⁹F NMR (DMSO-d₆) | NT² | NT² | conforms to |
| | | | structure |
| Purity (HPLC Area %) | 99.95% | 99.83% | 99.94% |
| Total impurities | 0.05% | 0.17% | 0.06% |
| Moisture (Karl | 3.9% | 3.9% | 3.9% |
| Fischer) | | | |
| Chloride ion | 5.9% | 5.9% | 6.0% |
| Residue on ignition | 0.15% | 0.15% | <0.1% |
| Endotoxin levels | NT² | NT² | <5 EU/mg |
| Bioburden: | NT² | NT² | <10 CFU/g |

¹Based upon test methods and target values in Table 1.
²Not tested

### Stability Testing

TABLE 8
Stability of a Representative Preparation
of Compound 298•HCl•H₂O

| | Condition 2: 25° C., 60% RH¹ | | | |
|---|---|---|---|---|
| Time | HPLC Assay² | Total | Potency Assay² | Water Content⁵ |
| (mos) | (% Purity³) | Impurities² (%) | (HPLC wt %) | (%) |
| 0 | 99.9 | 0.06 | 89.1 | 3.9 |
| 1 | NT⁴ | NT | NT | NT |
| 3 | 99.9 | 0.12 | 90.2 | 4.2 |
| 6 | 99.9 | 0.07 | 90.1 | 4.1 |
| 9 | 99.9 | 0.06 | 90.5 | 4.3 |
| 12 | 99.9 | 0.06 | 89.9 | 3.1 |
| 18 | 99.8 | 0.07 | 89.8 | 4.0 |
| | Condition 3: 40° C., 75% RH¹ | | | |
| Time | HPLC Assay² | Total | Potency Assay² | Water Content⁵ |
| (mos) | (% Purity³) | Impurities² (%) | (HPLC wt %) | (%) |
| 0 | 99.9 | 0.06 | 89.1 | 3.9 |
| 1 | 99.8 | 0.18 | 88.4 | 3.5 |
| 3 | 99.9 | 0.12 | 92.1 | 3.7 |
| 6 | 99.9 | 0.07 | 89.6 | 4.2 |

¹Relative humidity
²Method 4B
³By weight
⁴Not tested
⁵Method 4G

Appearance as a white powder remained unchanged over the 6 month period.
XRPD analysis of samples at both storage conditions after 6 months were still consistent with the standard pattern (see FIG. 9).

### Example 4

### Preparation of Compound 298.HCl.2H₂O

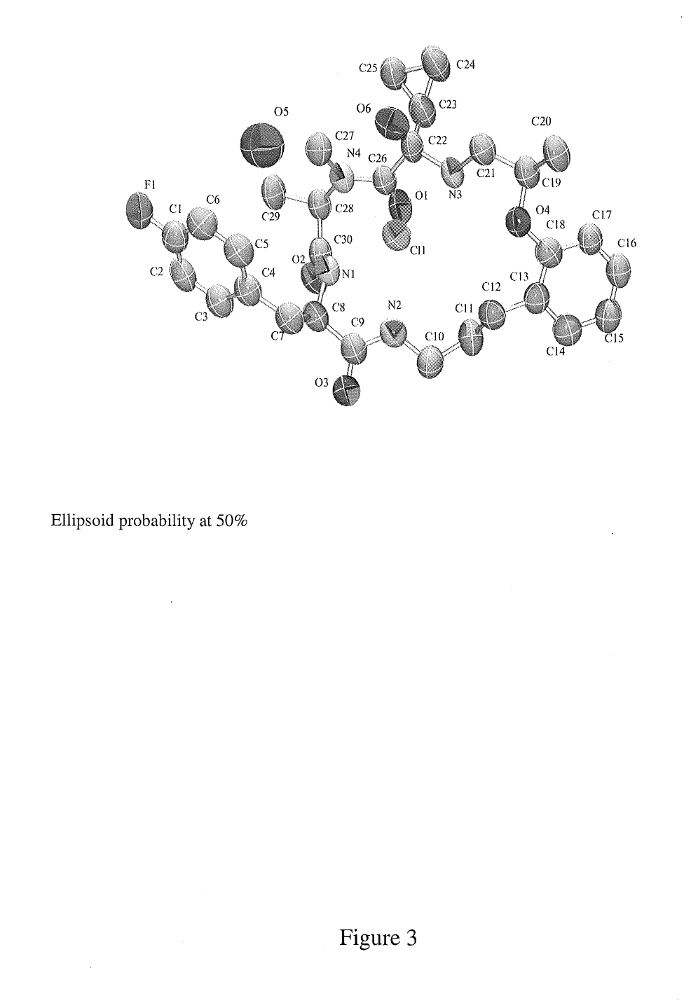

Amorphous compound 298.HCl was suspended in hot H₂O and methylethylketone (MEK) added dropwise using a Pasteur pipette until complete dissolution was observed. The solution was slowly cooled to room temperature using an oil bath (90° C.->25° C.) before being placed at 4° C., O/N. These crystals were collected with maintenance of temperature at 203±2 K and an X-ray structure taken rapidly. This structure confirmed the identity of compound 298.HCl.2H₂O salt and is provided as FIG. 3. Upon standing at room temperature, this solvate spontaneously loses one molecule of water to form compound 298.HCl.H₂O.

### Crystal Data and Structure Refinement for Compound 298.HCl.2H₂O

| Empirical formula | C₃₀H₄₄ClFN₄O₆ | |
|---|---|---|
| Formula weight | 611.14 | |
| Temperature | 203(2) K | |
| Wavelength | 1.54056 Å | |
| Crystal system | Orthorhombic | |
| Space group | p212121 | |
| Unit cell dimensions | a = 10.479(3) Å | α = 90°. |
| | b = 13.409(5) Å | β = 90°. |
| | c = 22.100(6) Å | γ = 90°. |
| Volume | 3105.2(16) Å³ | |
| Z | 4 | |
| Density (calculated) | 1.307 Mg/m³ | |
| Absorption coefficient | 1.543 mm⁻¹ | |
| F(000) | 1304 | |
| Crystal size | 0.50 × 0.30 × 0.30 mm³ | |
| Theta range for data | 3.85 to 69.78°. | |
| collection | | |
| Index ranges | 0 <= h <= 12, 0 <= | |
| | k <= 16, 0 <= 1 <= 26 | |
| Reflections collected | 3259 | |
| Independent reflections | 3259 [R(int) = 0.0000] | |
| Completeness to theta = | 98.3% | |
| 69.78° | | |
| Absorption correction | Empirical | |
| Max. and min. transmission | 0.6546 and 0.5125 | |
| Refinement method | Full-matrix least-squares | |
| | on F² | |
| Data/restraints/parameters | 3259/5/399 | |
| Goodness-of-fit on F² | 1.030 | |
| Final R indices [I > | R1 = 0.0741, wR2 = | |
| 2sigma(I)] | 0.1920 | |
| R indices (all data) | R1 = 0.0999, wR2 = | |
| | 0.2149 | |
| Absolute structure parameter | 0.02(4) | |
| Extinction coefficient | 0.0157(16) | |
| Largest diff. peak and hole | 0.358 and −0.426 e.Å⁻³ | |
| Intensity decay during | None | |
| data collection | | |
| Final BASF parameter | 0.02 | |

### Example 5

### Preparation of Compound 298.HCl.EtOH

The solvate was prepared by dissolving 100 mg of amorphous compound 298.HCl in hot H₂O/EtOH (1:1), then permitting the resulting solution to slowly cool to room temperature. The solution was placed at 4° C., O/N. The crystals of compound 298.HCl.EtOH were filtered and dried O/N at RT under vacuum (yield 85%).

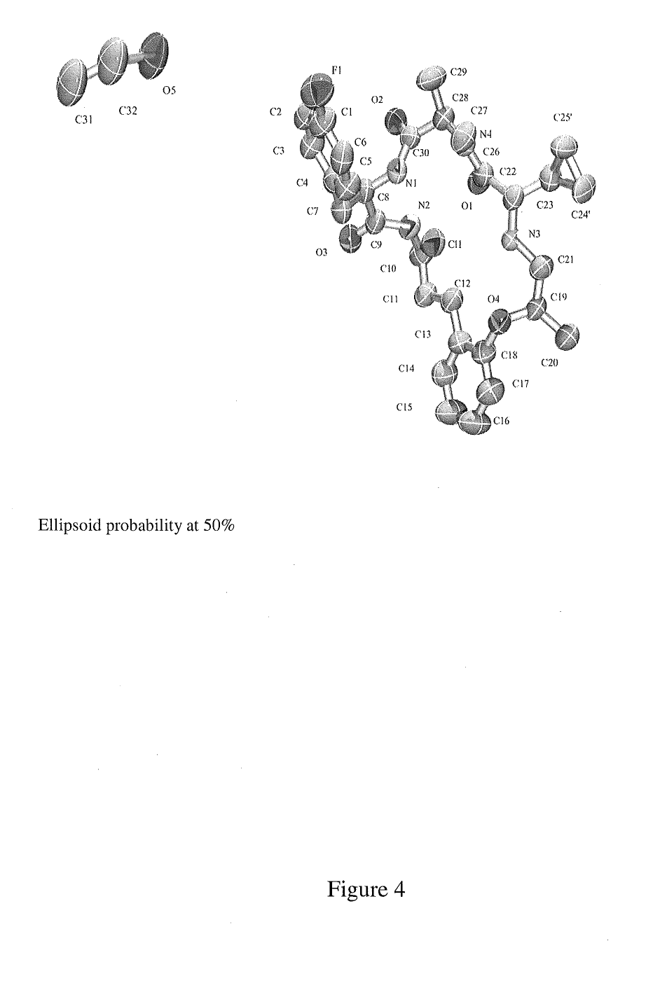

An X-ray structure of the crystals is shown as FIG. 4. The final absolute structure was determined by anomalous dispersion effects showing a twined crystal of the actual and the inverted structure. C24′ and C25′ group was disordered on two geometric sites, the final refined occupation was 54.5% for this group and 45.5% for C24 and C25. Only the major site is shown for clarity. The ethanol molecule was also disordered on two geometric sites, only the refined major site at 55.8% occupation is shown for the same reasons. C24, C24′, C25 and C25′ atoms were refined with equal thermal and bond restraints (EADP), the ethanol molecules were refined with similar thermal and bond restraints (SIMU, DELU).

Similar crystals were formed when compound 298 was initially dissolved in concentrated HCl/EtOH (1:1).

### Crystal Data and Structure Refinement for Compound 298.HCl.EtOH

| Empirical formula | C₃₂H₄₆ClFN₄O₅ | |
|---|---|---|
| Formula weight | 621.18 | |
| Temperature | 293(2) K | |
| Wavelength | 1.54176 Å | |
| Crystal system | Orthorhombic | |
| Space group | P212121 | |
| Unit cell dimensions | a = 13.062(5) Å | α = 90.00(5)° |
| | b = 14.438(7) Å | β = 90.00(4)° |
| | c = 17.627(11) Å | γ = 90.00(4)° |
| Volume | 3324(3) Å³ | |
| Z | 4 | |
| Density (calculated) | 1.241 Mg/m³ | |
| Absorption coefficient | 1.427 mm⁻¹ | |
| F(000) | 1328 | |
| Crystal size | 0.3 × 0.3 × 0.3 mm³ | |
| Theta range for data | 3.96 to 70.02°. | |
| collection | | |
| Index ranges | 0 <= h <= 15, 0 <= | |
| | k <= 17, 0 <= 1 <= 21 | |
| Reflections collected | 3418 | |
| Independent reflections | 3418 [R(int) = 0.0000] | |
| Completeness to theta = | 96.9% | |
| 70.02° | | |
| Absorption correction | Empirical | |
| Max. and min. transmission | 0.8390 and 0.7744 | |
| Refinement method | Full-matrix least-squares | |
| | on F² | |
| Data/restraints/parameters | 3418/38/414 | |
| Goodness-of-fit on F² | 0.967 | |
| Final R indices [I > | R1 = 0.0679, wR2 = | |
| 2sigma(I)] | 0.1697 | |
| R indices (all data) | R1 = 0.1143, wR2 = | |
| | 0.1973 | |
| Absolute structure parameter | 0.47(4) | |
| Extinction coefficient | 0.0068(8) | |
| Largest diff. peak and hole | 0.392 and −0.261 e.A⁻³ | |
| Intensity decay during | 2.7% | |
| data collection | | |
| Final BASF parameter | 0.47 | |

### Example 6

### Preparation of Salts of Compound 298 and Determination of their Solubility

To 20 mg (37.2 μmol, 1.0 eq) of compound 298 was added 0.5 mL of an aqueous solution of the acid (40.9 μmol, 1.1 eq). The resulting mixture was agitated on an orbital shaker for 72 h. The pH of the solution was then measured and the amount of dissolved salt determined by HPLC-CLND analysis. Table 9 summarizes the solubility results thus determined for 14 salts of compound 298.

TABLE 9
Solubilities of Representative Compound 298 Salts

| | Compound 298 | | Salt Solubility |
|---|---|---|---|
| Acid | Salt | pH at 72 hr | (mg/mL)¹ |
| Maleic | Maleate | 2.6 | 12.4 |
| Fumaric | Fumarate | 3.6 | 19.0 |
| Succinic | Succinate | 4.2 | 36.4 |
| Malonic | Malonate | 3.4 | 35.8 |
| L-Malic | Malate | 4.2 | 36.0 |
| Citric | Citrate | 3.6 | 30.4 |
| D-Tartaric | Tartrate | 3.5 | 4.4 |
| L-Lactic | Lactate | 4.6 | 37.0 |
| Formic | Formate | 3.8 | 35.8 |
| Methanesulfonic | Methanesulfonate | 2.1 | 14.8 |
| Ethanesulfonic | Ethanesulfonate | 1.8 | 36.4 |
| Sulfuric | Sulfate | 2.0 | 6.2 |
| Hydrochloric | Hydrochloride | 4.8 | 17.2 |
| Phosphoric | Phosphate | 3.3 | 35.0 |

¹Determined by HPLC-CLND quantitation

### Example 7

### Synthesis of Compound 298.Succinate Salt

To 500 mg of compound 298 free base in 10 mL of acetone was added 2 mL (1.1 eq) of a solution of succinic acid in water (prepared by dissolving 301 mg succinic acid in 5 mL water). The solution was agitated for 10 min, then the acetone evaporated in vacuo (rotary evaporator). The resulting aqueous solution was extracted with EtOAc (3×5 mL). The combined organic phase was dried over MgSO₄, filtered and the filtrate evaporated in vacuo. The residual solid thus obtained was dried O/N (vacuum pump). The salt was dissolved in a minimum amount of EtOAc, then heptane added to precipitate a white solid. The solid is triturated with heptane, collected by filtration and dried to yield 455 mg of compound 298.succinate. ¹H NMR was consistent with that expected for this salt (including a singlet at approximately 2.5 ppm).

Repetition of the above procedure starting from 100 mg of compound 298 free base provided 85 mg of the succinate salt. Repetition of the procedure starting from 3.0 g of compound 298 provided an essentially quantitative yield of compound 298.Succinate.

Crystalline compound 298.succinate was obtained by dissolving 50 mg of the amorphous material in 5 mL of Et₂O, then adding heptane dropwise until some turbidity was observed, but disappeared. The mixture was stored sealed at RT to afford long needles of 298.succinate after approximately 7 d. Alternatively, 100 mg of the amorphous material was dissolved in 13.5-15 mL Et₂O, heated to 40° C. (oil bath), then 1.5-2.5 mL heptanes added dropwise. The mixture was allowed to cool to RT, then stored at RT. Large, square transparent crystals were obtained. In other experiments, it was necessary to cool at −20° C. or permit slow Et₂O evaporation to effect crystallization.

mp: possible transition at 80°-90° C., decomposition at 155°-158° C.

### Example 8

### Synthesis of Compound 298.Malonate Salt

To 100 mg of compound 298 free base in 2 mL of acetone was added 0.4 mL (1.1 eq) of a solution of malonic acid in water (prepared by dissolving 269 mg malonic acid in 5 mL water). The solution was agitated for 10 min, then the acetone evaporated in vacuo (rotary evaporator). The resulting aqueous solution was extracted with EtOAc (3×5 mL). The combined organic phase was dried over MgSO₄, filtered and the filtrate evaporated in vacuo until about 2-3 mL of EtOAc remained, then heptane added to precipitate a white solid. The solvent was removed in vacuo to yield 85 mg of compound 298.malonate. If the solid was discolored, it was dissolved in a minimum amount of EtOAc, then heptane added to precipitate the salt, which is triturated with heptanes and collected by filtration. ¹H NMR was consistent with that expected for this salt (including a singlet at approximately 3.05 ppm). The salt was dried O/N (vacuum pump).

Repetition of the procedure starting from 3.0 g of compound 298 provided an essentially quantitative yield of compound 298.malonate.

mp: transition at 90°-110° C., decomposition at 165° C.

### Example 9

### Synthesis of Compound 298.Ethanesulfonate Salt

To 100 mg of compound 298 free base in 2 mL of acetone was added 0.4 mL (1.1 eq) of a solution of ethylsulfonic acid in water (prepared by dissolving 0.42 mL ethanesulfonic acid in 10 mL water). The solution was agitated for 10 min, then the acetone evaporated in vacuo (rotary evaporator). The resulting aqueous solution was extracted with EtOAc (3×5 mL). The combined organic phase was dried over MgSO₄, filtered and the filtrate evaporated in vacuo until about 2-3 mL of EtOAc remained, at which time a white solid precipitated. The salt was dried O/N (vacuum pump) to yield 75 mg of compound 298.ethanesulfonate. ¹H NMR was consistent with that expected for this salt [including the following characteristic peaks: 1.2 ppm (s), 2.1 ppm (br s), 8.2 ppm (m)].

Repetition of the procedure starting from 3.0 g of compound 298 provided 3.60 g (quantitative yield) of compound 298.ethanesulfonate.

mp: decomposition at 190°-195° C.

### Example 10

### Determination of the Solubility Stability for Salts and Solvates of Compound 298

To 5% dextrose in water (D5W), 5 mg and 20 mg of various compound 298 salts and solvates were added and pH established at 4, 5 or 6 with buffer. The samples were maintained at RT for 3 weeks with regular observations and solubility determinations (HPLC). The results are presented in Table 10.

TABLE 10
Stability of Solubility for Compound 298 Salts and Solvates

| Sample | Solubility (mg/mL) at pH 5 |
|---|---|
| Compound 298•HCl•H₂O | 5 |
| Compound 298•HCl•EtOH | 8 |
| Compound 298•HCl amorphous | 7 |
| Compound 298•succinate | 15-20 |

For all samples, solubility remained stable, or even increased slightly over the three week period. No precipitation was observed.

### Example 11

### Preparation of a Representative Pharmaceutical Composition of 298.HCl H₂O

A formulation of compound 298.HCl H₂O suitable for use as a pharmaceutical can be prepared utilizing the following procedure. Batch size can vary; the procedure for a 30 L batch is described.
1. Add approximately 25.0 L of sterile water for injection into a tared 40 L glass carboy with mixing.
2. Add 1363.68 g of dextrose anhydrous into the glass carboy and mix until dissolved.
3. Add 17.04 mL of glacial acetic acid to the solution from Step 2 and mix for a minimum of 5 min.
4. Record the pH of the formulation.
5. Adjust the pH of formulation to pH 4.5±0.2 with 1 N NaOH (aq) solution.
6. After this pH is attained, monitor the pH of the solution by regular measurements until it has stabilized.
7. Once the pH has stabilized, add 33.72 g of compound 298.HCl.H₂O to the glass carboy.
8. Agitate until complete dissolution is observed (approximately 1 h).
9. Add sterile water for injection to attain a final batch weight of 30.41 kg (density of 1.0136 g/mL).
10. Sterile filter the formulation through a 0.45 μm Durapore Millipak pre-filter and a 0.22 μm Durapore Millipak filter.
11. Perform in-process testing of the appearance and pH of the formulation.
12. Aseptically fill 10 mL glass vials with the formulation to a target weight of 9.37±0.19 g (approximately 9.5 mL).
13. Stopper and seal the vials, then inspect each.
   The resulting pharmaceutical composition can be analyzed as summarized in Table 11, with the expected results shown.

TABLE 11
Representative Analytical Tests for Compound
298•HCl•H₂O Pharmaceutical Composition

| Test | Method | Target Result |
|---|---|---|
| Appearance | Method 4O | Clear, colorless solution |
| | | with no visual particulates |
| Identification | HPLC (Method 4P) | The same retention time as |
| | | that of reference standard |
| Potency Assay | HPLC (Method 4P) | 90.0 to 110.0% (as free |
| | | base) |
| Degradation Product | HPLC (Method 4P) | Each degradant ≦0.5% |
| Assay | | Total degradants ≦2% |
| pH | Method 4Q | 4.0 to 5.0 pH units |
| Osmolality | Method 4R | 250 to 330 mOsmol/kg |
| Particulate Matter | Method 4S | USP requirements |
| Endotoxin | Method 4T | ≦0.20 EU/mL |
| Sterility | Method 4U | USP requirements |

The analysis of a representative pharmaceutical composition of compound 298.HCl.H₂O prepared using Example 11 is listed in Table 12. The stability of the pharmaceutical composition is presented in Table 13.

TABLE 12
Analysis of Pharmaceutical Composition of Example 11

| Test | Assay Method | Target Results | Actual Results |
|---|---|---|---|
| Appearance | Method 4O | Clear, colorless | Pass |
| | | solution with no | |
| | | visual particulates | |
| Identification | Method 4P | The same retention | Pass |
| | | time as that of | |
| | | reference standard | |
| Potency | Method 4P | 90.0 to 110.0% (as | 102.9% |
| Assay | | free base) | |
| Degradation | Method 4P | Each ≦0.5%^{a} | <0.1%^{b} |
| Product | | Total ≦2%^{a} | <0.1%^{b} |
| Assay | | | |
| pH | Method 4Q | 4.0 to 5.0 pH units | 4.5 |
| Osmolality | Method 4R | 250 to 330 mOsmol/kg | 283 mOsmol/kg |
| Particulate | Method 4S | USP requirements | Pass |
| Matter | | | |
| Endotoxin | Method 4T | ≦0.20 EU/mL | <0.18 EU/mL |
| Sterility | Method 4U | USP requirements | Pass |

^{a}Report results of each single degradant over 0.1% and the sum total of degradates.
^{b}No degradant observed over the threshold 0.1%. Only impurities already in active ingredient were observed. Assigned purity by HPLC-UV (λ = 230 nm) is 99.6%.

TABLE 13
Stability of a Representative Pharmaceutical
Composition of Example 11

| | Condition 1: 5° C. | | | |
|---|---|---|---|---|
| Time | Potency Assay | Total | | Particulate Matter³ |
| (mos) | (HPLC %)¹ | Impurities (%)¹ | pH² | (counts) |
| Target | 90.0-110.0 | ≦2.0 | 4.0-5.0 | ≧10 μm: <6000 |
| | | | | ≧25 μm: <600 |
| 0 | 98.1 | 0.42 | 4.40 | 10 μm: 24 |
| | | | | 25 μm: 1 |
| 3 | 98.1 | 0.41 | 4.46 | 10 μm: 10 |
| | | | | 25 μm: 1 |
| 4 | 101.5 | 0.48 | 4.33 | NT^{b} |
| 6 | 102.6 | 0.49 | 4.42 | 10 μm: 10 |
| | | | | 25 μm: 0 |
| 9 | 102.2 | 0.43 | 4.50 | 10 μm: 11 |
| | | | | 25 μm: 0 |
| 12 | 102.3 | 0.43 | 4.03^{a} | 10 μm: 6 |
| | | | | 25 μm: 2 |
| 18 | 99.9 | 0.10 | 4.44 | 10 μm: 13 |
| | | | | 25 μm: 3 |
| 24 | 102.9 | 0.43 | 4.09^{a} | 10 μm: 8 |
| | | | | 25 μm: 0 |

| | Condition 2: 25° C., 60% RH^{c} | | | |
|---|---|---|---|---|
| Time | Potency Assay | Total | | Particulate Matter³ |
| (mos) | (HPLC %)¹ | Impurities (%)¹ | pH² | (counts) |
| 0 | 100.3 | 0.42 | 4.40 | 10 μm: 24 |
| | | | | 25 μm: 1 |
| 1 | 102.6 | 0.41 | 4.27 | 10 μm: 29 |
| | | | | 25 μm: 12 |
| 3 | 97.0 | 0.41 | 4.46 | 10 μm: 8 |
| | | | | 25 μm: 0 |
| 4 | 100.7 | 0.46 | 4.33 | NT^{b} |
| 6 | 108.0 | 0.46 | 4.44 | 10 μm: 6 |
| | | | | 25 μm: 0 |
| 9 | 101.4 | 0.43 | 4.48 | 10 μm: 21 |
| | | | | 25 μm: 0 |
| 12 | 100.2 | 0.42 | 4.05^{a} | 10 μm: 16 |
| | | | | 25 μm: 1 |
| 18 | 100.7 | 0.10 | 4.42 | 10 μm: 7 |
| | | | | 25 μm: 1 |
| 24 | 100.7 | 0.41 | 4.07^{a} | 10 μm: 31 |
| | | | | 25 μm: 0 |
| | Condition 3: 40° C., 75% RH^{c} | | | |
| Time | Potency Assay | Total | | Particulate Matter³ |
| (mos) | (HPLC %)¹ | Impurities (%)¹ | pH² | (counts) |
| 0 | 100.3 | 0.42 | 4.40 | 10 μm: 24 |
| | | | | 25 μm: 1 |
| 1 | 101.2 | 0.40 | 4.30 | 10 μm: 20 |
| | | | | 25 μm: 13 |
| 3 | 98.2 | 0.53 | 4.48 | 10 μm: 98 |
| | | | | 25 μm: 31 |
| 4 | 101.9 | 0.63 | 4.33 | NT^{b} |
| 6 | 105.2 | 0.73 | 4.44 | 10 μm: 35 |
| | | | | 25 μm: 5 |

¹Method 4P
²Method 4Q
³Method 4S
^{a}Measured using standard electrode, all other measurements with microelectrode.
^{b}Not tested
^{c}Relative humidity

Appearance (Method 4O) as a clear, colorless solution remained unchanged over the entire 24 month period in all samples examined.
Testing at 6, 12, 18, 24 months showed that all samples tested remained sterile (Method 4U).
Testing of Condition 3 was only conducted for 6 months.

### Pharmacokinetic Analysis

A pharmacokinetic analysis after intravenous administration of single and multiple doses of a pharmaceutical composition of Example 11 to healthy human volunteers has been reported. (Lasseter, K. C.; Shaughnessy, L.; Cummings, D.; et al. J. Clin. Pharmacol. 2008, 48, 193-202.)

### Example 12

### Preparation of a Representative Pharmaceutical Composition of 298.HCl H₂O

A formulation of 298.HCl.H₂O suitable for use as a pharmaceutical product can be prepared utilizing the following procedure which is a variation of that of Example 11. Batch size can vary; the procedure for an approximately 50 L batch is described.
1. Add approximately 45.0 L of sterile water for injection (WFI) into a tared clean 50 L glass carboy with mixing.
2. Add 2272.8 g of dextrose anhydrous into the glass carboy and mix until dissolved.
3. Add 28.40 mL of glacial acetic acid to the solution from Step 2 and mix for a minimum of 5 min.
4. Record the pH of the formulation.
5. Adjust the pH of formulation to pH 4.5±0.2 with 1 N NaOH (aq) solution (requires approximately 210 mL).
6. After this pH is attained, monitor the pH of the solution by regular measurements until it has stabilized.
7. Once the pH has stabilized, add 56.20 g of compound 298.HCl.H₂O to the glass carboy.
8. Agitate until complete dissolution is observed (approximately 1-2 h).
9. Add sterile WFI to attain a final batch weight of 50.68 kg (density of 1.0136 g/mL).
10. Sterile filter the formulation through two 0.22 μm Durapore Millipak filters.
11. Perform in-process testing of the appearance and pH of the formulation.
12. Aseptically fill 10 mL glass vials with the formulation containing 298.HCl monohydrate drug product to a target weight of 9.63±0.19 g.
13. Stopper and seal the vials, then inspect each.
   The analysis of a representative pharmaceutical composition of compound 298.HCl.H₂O prepared using Example 12 is listed in Table 14. The stability of two separate preparations of the pharmaceutical composition is presented in Tables 15 and 16.

TABLE 14
Representative Analyses of Pharmaceutical Compositions of Example 12

| | Assay | | Results | Results |
|---|---|---|---|---|
| Test | Method | Specifications | (Batch 1) | (Batch 2) |
| Appearance | Method 4O | Clear, colorless | Conforms | Conforms |
| | | solution with no | | |
| | | visual particulates | | |
| Identification | Method 4P | The same retention | Conforms | Conforms |
| | | time as that of | | |
| | | reference standard | | |
| Potency Assay | Method 4P | 90.0 to 110.0% | 101.0% | 103.1% |
| | | (Label claim as | | |
| | | free base) | | |
| Degradation | Method 4P | Each ≦0.5%^{a} | <0.1%^{b} | <0.1%^{b} |
| Product Assay | | Total ≦2%^{a} | <0.1%^{b} | <0.1%^{b} |
| pH | Method 4Q | 4.0 to 5.0 pH units | 4.4 | 4.3 |
| Osmolality | Method 4R | 250 to 330 mOsmol/kg | 258 mOsmol/kg | 257 mOsmol/kg |
| Particulate | Method 4S | USP requirements | Pass | Pass |
| Matter | | | | |
| Endotoxin | Method 4T | ≦0.20 EU/mL | <0.20 EU/mL | <0.20 EU/mL |
| Sterility | Method 4U | USP requirements | Pass | Pass |

^{a}Report results of each single degradant over 0.1% and the sum total of degradants.
^{b}No degradant observed over the threshold 0.1%. Only impurities already in active ingredient were observed. Assigned purity by HPLC-UV (λ = 230 nm) is 99.5%.

TABLE 15
Stability of a Representative Pharmaceutical
Composition of Example 12 (Batch 1)

| | Condition 1: 5° C. | | | |
|---|---|---|---|---|
| Time | Potency Assay | Total | | Particulate Matter³ |
| (mos) | (HPLC %)¹ | Impurities (%)¹ | pH² | (counts) |
| Target | 90.0-110.0 | ≦2.0 | 4.0-5.0 | ≧10 μm: <6000 |
| | | | | ≧25 μm: <600 |
| 0 | 101.0 | 0.47 | 4.4 | 10 μm: 6 |
| | | | | 25 μm: 0 |
| 3 | 101.0 | 0.43 | 4.6 | 10 μm: 0 |
| | | | | 25 μm: 0 |
| 6 | 100.5 | 0.45 | 4.5 | 10 μm: 1 |
| | | | | 25 μm: 0 |
| 9 | 101.2 | 0.45 | 4.5 | 10 μm: 0 |
| | | | | 25 μm: 0 |
| 12 | 100.8 | 0.44 | 4.5 | 10 μm: 1 |
| | | | | 25 μm: 0 |
| 18 | 100.4 | 0.44 | 4.6 | 10 μm: 1 |
| | | | | 25 μm: 1 |
| 24 | 103.2 | 0.44 | 4.5 | 10 μm: 1 |
| | | | | 25 μm: 0 |

| | Condition 2: 25° C., 60% RH^{c} | | | |
|---|---|---|---|---|
| Time | Potency Assay | Total | | Particulate Matter³ |
| (mos) | (HPLC %)¹ | Impurities (%)¹ | pH² | (counts) |
| 0 | 101.0 | 0.47 | 4.4 | 10 μm: 6 |
| | | | | 25 μm: 0 |
| 3 | 101.0 | 0.44 | 4.5 | 10 μm: 1 |
| | | | | 25 μm: 1 |
| 6 | 101.4 | 0.44 | 4.6 | 10 μm: 3 |
| | | | | 25 μm: 0 |
| 9 | 101.0 | 0.46 | 4.5 | 10 μm: 1 |
| | | | | 25 μm: 1 |
| 12 | 101.4 | 0.45 | 4.5 | 10 μm: 1 |
| | | | | 25 μm: 1 |
| 18 | 100.7 | 0.45 | 4.6 | 10 μm: 1 |
| | | | | 25 μm: 0 |
| 24 | 102.9 | 0.56 | 4.5 | 10 μm: 1 |
| | | | | 25 μm: 0 |
| | Condition 3: 40° C., 75% RH^{c} | | | |
| Time | Potency Assay | Total | | Particulate Matter³ |
| (mos) | (HPLC %)¹ | Impurities (%)¹ | pH² | (counts) |
| 0 | 101.0 | 0.47 | 4.4 | 10 μm: 6 |
| | | | | 25 μm: 0 |
| 3 | 100.3 | 0.44 | 4.5 | 10 μm: 1 |
| | | | | 25 μm: 0 |
| 6 | 100.8 | 0.72 | 4.6 | 10 μm: 9 |
| | | | | 25 μm: 0 |

¹Method 4P
²Method 4Q
³Method 4S
^{a}Measured using standard electrode, all other measurements with microelectrode.
^{b}Not tested
^{c}Relative humidity

Appearance (Method 4O) as a clear, colorless solution remained unchanged over the entire 24 month period in all samples examined.
Testing at 6, 12, 18, 24 months showed that all samples tested remained sterile (Method 4U). Testing of only Condition 3 at 3 months showed that the sample was still sterile. Testing of Condition 3 was conducted for 6 months only.

TABLE 16
Stability of a Representative Pharmaceutical
Composition of Example 12 (Batch 2)

| | Condition 1: 5° C. | | | |
|---|---|---|---|---|
| Time | Potency Assay | Total | | Particulate Matter³ |
| (mos) | (HPLC %)¹ | Impurities (%)¹ | pH² | (counts) |
| Target | 90.0-110.0 | ≦2.0 | 4.0-5.0 | ≧10 μm: <6000 |
| | | | | ≧25 μm: <600 |
| 0 | 103.1 | 0.47 | 4.3 | 10 μm: 7 |
| | | | | 25 μm: 0 |
| 3 | 100.1 | 0.44 | 4.6 | 10 μm: 5 |
| | | | | 25 μm: 0 |
| 6 | 100.9 | 0.45 | 4.6 | 10 μm: 1 |
| | | | | 25 μm: 1 |
| 9 | 100.4 | 0.44 | 4.6 | 10 μm: 4 |
| | | | | 25 μm: 1 |
| 12 | 101.2 | 0.44 | 4.6 | 10 μm: 13 |
| | | | | 25 μm: 3 |
| 18 | 101.2 | 0.60 | 4.6 | 10 μm: 3 |
| | | | | 25 μm: 2 |
| 24 | 101.1 | 0.44 | 4.5 | 10 μm: 1 |
| | | | | 25 μm: 0 |

¹Method 4P
²Method 4Q
³Method 4S
^{a}Measured using standard electrode, all other measurements with microelectrode.
^{b}Not tested
^{c}Relative humidity

Appearance (Method 4O) as a clear, colorless solution remained unchanged over the entire 24 month period in all samples examined.
Testing at 6, 12, 18, 24 months showed that all samples tested remained sterile (Method 4U).

### Example 13

### Preparation of a Representative Pharmaceutical Composition of Compound 298.HCl.H₂O

Another preparation of a composition of compound 298.HCl.H₂O suitable for pharmaceutical use is described below. Prior to the stepwise operations outlined, the stainless steel tanks and the connection pipelines between the tanks and to the filling machine are typically sterilized in place.
1. Water for injection (WFI) equal to approximately 85% of the final target weight was loaded into a 200 L stainless steel compounding tank equipped with a magnetic stirrer at a temperature of 25±5° C. The mixer was started so that there was continuous mixing during subsequent additions.
2. To this was added acetic acid (100%) and the pH measured.
3. The pH was then adjusted to 4.5±0.2 using 1 N NaOH.
4. When this pH was reached, the entire amount of dextrose (anhydrous) required was added with continuous mixing.
5. After complete dissolution of the dextrose, compound 298.HCl.H₂O was added and the mixture agitated for 1 h. If necessary, longer mixing is performed to ensure complete dissolution of the macrocycle prior to continuing.
6. Upon complete dissolution, the remaining quantity of WFI was added.
7. The solution was pre-filtered through a sterilizing Pall 0.22 μm cartridge filter (for example product no. MCY4440DFLPH4) using nitrogen pressure. (Note that prior to and after use all the solution filters were wetted with WFI and integrity tested using an appropriate method, such as the Bubble Point test or the Forward Flow test.)
8. The solution can be stored under nitrogen until ready for filling into appropriate containers, such as vials.
9. For filling, the pre-filtered solution was sterilized by pressure filtration through two 0.22 μm Pall filters (for example product no. KA3DFLP1) into the filling reservoir.
10. For filling vials with the pharmaceutical composition, a Bosch FLC 3080 filling/stoppering machine (or similar) was employed. As an example, a 10.5 mL target fill volume with 10 mL glass vials was prepared.
   A batch size of approximately 170 L of a pharmaceutical composition of compound 298.HCl.H₂O (2 mg/mL) has been prepared using the procedure of Example 13, although batch sizes smaller or larger can also be prepared with the method. The quantities of components used for this batch are shown in Table 17. Test results for this representative pharmaceutical composition (2 mg/mL) are presented in Table 18 and the stability of the representative pharmaceutical composition is presented in Table 19.

TABLE 17
Quantities of Components for Preparation of a Representative
Pharmaceutical Composition of Example 13

| Component | Quantity | |
|---|---|---|
| 298•HCl•H₂O | 340.0 | g |
| Dextrose (anhydrous) | 8.16 | kg |
| Glacial acetic acid | 0.107 | L |
| Sodium hydroxide (used to prepare 1.0N solution) | 40 | g |
| Water for injection | 164.36 | L |

TABLE 18
Representative Analysis of a Pharmaceutical Composition of Example 13

| Test | Assay Method | Target Result or Range | Results |
|---|---|---|---|
| Appearance | Method 4O | Clear, colorless | Clear, colorless |
| | | solution | solution |
| Identification | Method 4P | The same retention | The same retention |
| | | time as that of | time as that of |
| | | standard | standard |
| Potency Assay (as free base) | Method 4P | 90.0 to 110.0% | 100.4% |
| Total Impurities (area %) | Method 4P | ≦1.5% | <0.10% |
| pH | Method 4Q | 4.3-4.7 | 4.5 |
| Osmolarity | Method 4R | 270 to 330 mOsmol/kg | 301 mOsmol/kg |
| Particulate | Method 4S | ≧10 μm: <6000 | ≧10 μm: 3 |
| Matter (counts) | | ≧25 μm: <600 | ≧25 μm: 3 |
| Bacterial Endotoxins | Method 4T | ≦0.20 | <0.16 |
| (EU/mL) | | | |
| Sterility | Method 4U | Sterile | Sterile |

TABLE 19
Stability of a Representative Pharmaceutical
Composition of Example 13

| | Condition 1: 25° C., 60% RH^{b} | | | |
|---|---|---|---|---|
| Time | Potency Assay | Total | | Particulate Matter³ |
| (mos) | (HPLC %)¹ | Impurities (%)¹ | pH² | (counts) |
| 0 | 100.4 | <0.1 | 4.5 | 10 μm: 3 |
| | | | | 25 μm: 3 |
| 3 | 102.6 | <0.1 | 4.5 | 10 μm: NT⁴ |
| | | | | 25 μm: NT |
| 6 | 100.4 | <0.1 | 4.5 | 10 μm: 11 |
| | | | | 25 μm: 4 |
| 9 | 102.3 | <0.1 | 4.5 | 10 μm: NT |
| | | | | 25 μm: NT |
| 12 | 100.6 | <0.1 | 4.4 | 10 μm: 20 |
| | | | | 25 μm: 1 |
| | Condition 2: 40° C., 75% RH^{b} | | | |
| Time | Potency Assay | Total | | Particulate Matter³ |
| (mos) | (HPLC %)¹ | Impurities (%)¹ | pH² | (counts) |
| 0 | 100.4 | <0.1 | 4.5 | 10 μm: 3 |
| | | | | 25 μm: 3 |
| 3 | 102.1 | 0.15 | 4.5 | 10 μm: NT⁴ |
| | | | | 25 μm: NT |
| 6 | 99.7 | 0.30 | 4.5 | 10 μm: 5 |
| | | | | 25 μm: 2 |

¹Method 4P
²Method 4Q
³Method 4S
⁴Not tested
^{a}Measured using standard electrode, all other measurements with microelectrode.
^{b}Relative humidity

Appearance (Method 4O) as a clear, colorless solution remained unchanged over the entire 12 month period in all samples examined.
Testing of Condition 1 at 6 and 12 months showed that all samples tested remained sterile (Method 4U). Testing of Condition 2 at 6 months showed that the sample was still sterile. Testing of Condition 2 was conducted for 6 months only.

### Example 14

### Preparation and Pharmacokinetics (PK) of Representative Pharmaceutical Compositions of Compound 298.HCl.H₂O for Subcutaneous Administration

An aqueous solution of compound 298.HCl.H₂O in acetate buffer and 5% Dextrose in Water (D5W) was prepared at a concentration of 0.4 mg/mL (as equivalent to compound 298 free base), with a final pH of 4.5 and designated as Formulation A. It was prepared by diluting 4 mL of the composition of Example 11 at 1 mg/mL (as compound 298 free base) in 10 mM acetate buffer in D5W with 6 mL of D5W.

Solutions of various concentrations of compound 298.HCl.H₂O in 12% N-methylpyrrolidone (NMP) and 0.25% benzyl alcohol in D5W were prepared by dissolving compound 298.HCl.H₂O in NMP with approximately 5 min stirring, followed by addition of benzyl alcohol and subsequently D5W. The resulting Formulations B-1, B-2 and B-3 were at 0.4, 1.6 and 6.4 mg/mL, which correspond to 0.36, 1.45 and 5.81 mg/mL of compound 298 free base (MW=538.65 g/mol), respectively.

Formulations A, B-1, B-2 and B-3 were administered to male Sprague-Dawley rats subcutaneously (sc) at a dosing volume of 5 mL/kg. Details of the compound administration are presented in Table 20.

TABLE 20
Administration Details for Representative Pharmaceutical
Compositions of Compound 298•HCl•H₂O

| | Pharma- | | | Dose | Dose | Dose |
|---|---|---|---|---|---|---|
| Dose | ceutical | Dose | No. of | Level | Volume | Conc. |
| Group | Composition | Route | Doses | (mg/kg) | (mL/kg) | (mg/mL) |
| 1 | Formulation | sc | 1 | 2.0 | 5 | 0.4 |
| | A | | | | | |
| 2 | Formulation | sc | 3 | 1.8 | 5 | 0.36 |
| | B-1 | | | | | |
| 3 | Formulation | sc | 3 | 7.3 | 5 | 1.45 |
| | B-2 | | | | | |
| 4 | Formulation | sc | 3 | 29.1 | 5 | 5.81 |
| | B-3 | | | | | |

Note: Dose levels are in mg/kg of compound 298 free base.

### Blood Sampling Protocol

Blood samples (approximately 300 μL) were collected at the following times: pre-dose, 5, 15, 30, 60, 120, 240, 360 and 480 min after sc administration. Blood was collected via the JVC into tubes containing sodium EDTA (NaEDTA) and placed on ice until centrifugation. Samples were centrifuged at 13,000 rpm for 5 min with the temperature maintained at 4° C. Plasma was separated and stored frozen on dry ice prior to analysis. The compound 298 concentrations in rat plasma samples at each sampling time-point were determined by LC-MS-MS.

### Calculations

The pharmacokinetic (PK) parameters for each animal were calculated using non-compartmental modeling (extravascular input model) and WinNonlin software (version 5.2, Pharsight). The half-life (t_{1/2}), Area Under the Concentration Versus Time Curve from time 0 to the last quantifiable point (AUC₀₋ₜ) and to infinity (AUC_{0-∞}) were calculated and the observed Cₘₐₓ and Tₘₐₓ tabulated for each animal and dose group. The average Cₘₐₓ observed after the subcutaneous administration of compound 298 at 2 mg/kg was about 1.6 μg/mL (range 1.2-1.8 μg/mL) for Formulation A and 1.5 μg/mL (range 1.3-1.7 μg/mL) for Formulation B. The average Cₘₐₓ observed at higher doses of compound 298.HCl.H₂O in Formulation B was about 2.6 μg/mL (range 2.1-2.9 μg/mL) and 3.3 μg/mL (range 2.8-3.7 μg/mL) after a dose of 7 or 29 mg/kg, respectively. The increase in Cₘₐₓ was less than dose-proportional after subcutaneous administration perhaps due to rate-limited absorption from the subcutaneous compartment. Nevertheless, the plasma levels were maintained at a high and stable level for a long period after drug administration suggesting high and sustained plasma exposure after subcutaneous administration of the compound. The terminal elimination rate constant was calculated for the purpose of obtaining the extrapolated AUC_{0-∞} values. The AUC_{0-∞} increased proportionally with dose in this study indicating linear absorption from the subcutaneous compartment within the dose range tested. The PK parameters determined for these representative pharmaceutical compositions are summarized in Table 21.

TABLE 21
Pharmacokinetic Parameters for Representative Pharmaceutical
Compositions of Compound 298•HCl•H₂O after Subcutaneous
Administration to Rats

| Dose | Dose | | | |
|---|---|---|---|---|
| (mg/kg) | Formulation | Parameter | Average | S.D.¹ |
| 2 | Formulation A | Cₘₐₓ (ng/mL) | 1558 | 295 |
| | | Tₘₐₓ (min) | 60 | 0 |
| | | t_{1/2} (min) | 41 | 4 |
| | | AUC₀₋ₜ (ng · min/mL) | 252786 | 30141 |
| | | AUC_{0-∞} (ng · min/mL) | 254237 | 30752 |
| 2 | Formulation B-1 | Cₘₐₓ (ng/mL) | 1520 | 217 |
| | | Tₘₐₓ (min) | 25 | 9 |
| | | t_{1/2} (min) | 67 | 9 |
| | | AUC₀₋ₜ (ng · min/mL) | 259078 | 26240 |
| | | AUC_{0-∞} (ng · min/mL) | 261835 | 24792 |
| 7 | Formulation B-2 | Cₘₐₓ (ng/mL) | 2614 | 415 |
| | | Tₘₐₓ (min) | 40 | 17 |
| | | t_{1/2} (min) | 96 | 18 |
| | | AUC₀₋ₜ (ng · min/mL) | 738030 | 143057 |
| | | AUC_{0-∞} (ng · min/mL) | 790040 | 173769 |
| 29 | Formulation B-3 | Cₘₐₓ (ng/mL) | 3263 | 488 |
| | | Tₘₐₓ (min) | 70 | 46 |
| | | t_{1/2} (min) | 571 | 55 |
| | | AUC₀₋ₜ (ng · min/mL) | 1233688 | 166664 |
| | | AUC_{0-∞} (ng · min/mL) | 2912933 | 422670 |

¹S.D. = standard deviation

The foregoing is illustrative of the present invention, and is not to be construed as limiting thereof. The invention is defined by the following claims, with equivalents of the claims to be included therein.

What is claimed is:

1. A solvate of a salt of a macrocyclic compound with the following structure: 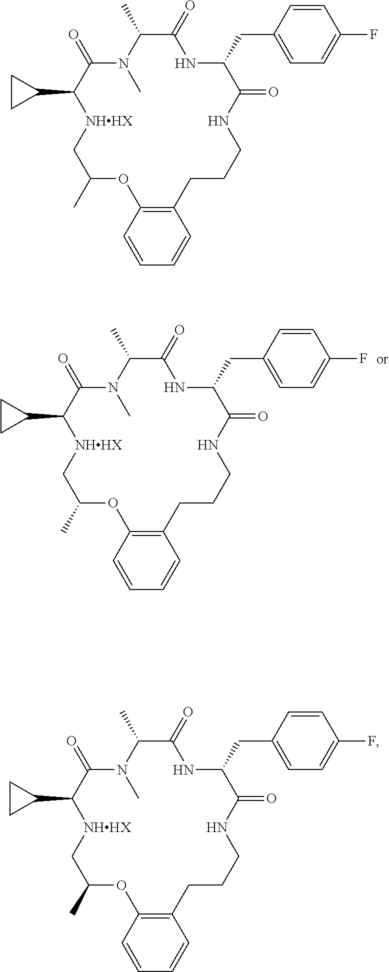
wherein HX is selected from the group consisting of hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, an amino acid, an aromatic acid and a sulfonic acid.

2. The solvate of claim 1, wherein HX is hydrochloric acid, succinic acid, malonic acid or ethylsulfonic acid.

3. The solvate of claim 1, wherein the solvate is an amorphous form.

4. The solvate of claim 1, wherein the solvate is a crystalline form.

5. The solvate of claim 1, wherein the solvate is an ethanolate, hydrate or monohydrate.

6. The solvate of claim 1, wherein the solvate is monohydrochloride monohydrate, monohydrochloride dihydrate or monohydrochloride monoethanolate.

7. A polymorphic form of a solvate of claim 1.

8. The polymorphic form of claim 7, wherein HX is hydrochloric acid.

9. The polymorphic form of claim 7, wherein the X-ray powder diffraction pattern conforms to that of FIG. 9.

10. The polymorphic form of claim 7, wherein the X-ray powder diffraction pattern contains characteristic peaks expressed in degrees 2θ at about 7.9, 9.3, 15.9, 17.9, and 23.9.

11. The polymorphic form of claim 7, wherein the X-ray powder diffraction pattern contains characteristic peaks expressed in degrees 2θ at about 7.7, 7.9, 9.3, 11.4, 11.5, 13.3, 14.5, 15.9, 16.2, 16.8, 17.2, 17.6, 17.9, 19.7, 21.6, 22.3, 22.6, 23.2, 23.9, 24.8, 25.3, 26.2, 26.6, and 26.9.

12. A process for preparing the polymorphic form of claim 7, the process comprising:
(a) dissolving a macrocyclic compound with the structure 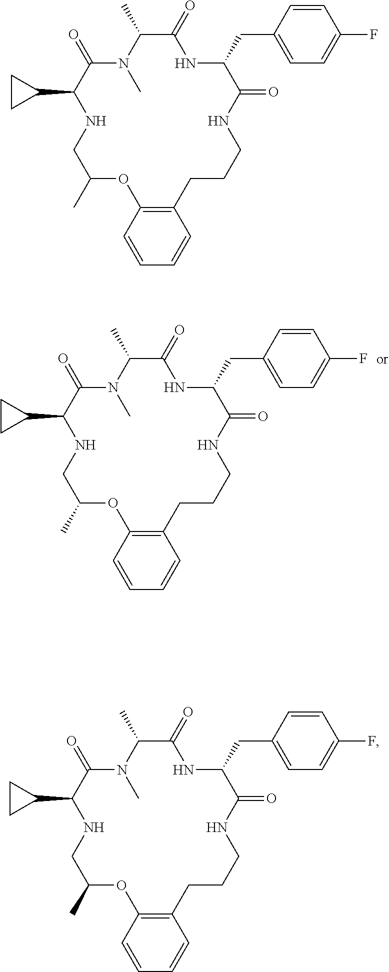
in a solution of an alcohol to form solution A;
(b) adding an acid, HX, to solution A to form acidified solution A, wherein HX is selected from the group consisting of hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, an amino acid, an aromatic acid and a sulfonic acid;
(c) optionally cooling acidified solution A;
(d) separating a precipitated salt from acidified solution A;
(e) dissolving the precipitated salt from (d) in a hot mixture of an alcohol and water to form solution B;
(f) cooling solution B;
(g) separating a precipitated salt from solution B;
(h) dissolving the precipitated salt from (g) in a hot mixture of a ketone solvent and water to form solution C;
(i) cooling solution C to ambient temperature or below; and
(j) separating a precipitated salt from solution C.

13. The process of claim 12, wherein the alcohol is ethanol.

14. The process of claim 12, wherein HX is hydrochloric acid.

15. The process of claim 12, wherein the ketone solvent is methyl ethyl ketone (2-butanone).

16. The process of claim 12, wherein the hot mixture of a ketone solvent and water is a mixture of methyl ethyl ketone and water in a 1:4 proportion.

17. A pharmaceutical composition comprising:
(a) a solvate of claim 1; and
(b) a pharmaceutically acceptable carrier, excipient or diluent.

18. The pharmaceutical composition of claim 17, wherein the pharmaceutically acceptable carrier, excipient or diluent is a buffer.

19. The pharmaceutical composition of claim 18, wherein the buffer is an acetate buffer.

20. The pharmaceutical composition of claim 17, wherein the pharmaceutically acceptable carrier, excipient or diluent is a tonicity agent.

21. The pharmaceutical composition of claim 20, wherein the tonicity agent is saline, sodium chloride or dextrose.

22. The pharmaceutical composition of claim 17, wherein the pharmaceutically acceptable carrier, excipient or diluent comprises a buffer and a tonicity agent.

23. The pharmaceutical composition of claim 22, wherein the buffer is an acetate buffer and the tonicity agent is saline, sodium chloride or dextrose.

24. A pharmaceutical composition comprising:
(a) a polymorphic form of claim 7; and
(b) a pharmaceutically acceptable carrier, excipient or diluent.

25. A pharmaceutical composition comprising:
(a) a polymorphic form of claim 7;
(b) 10 nM acetate; and
(c) 5% dextrose in water.

26. A salt of a macrocyclic compound with the following structure: 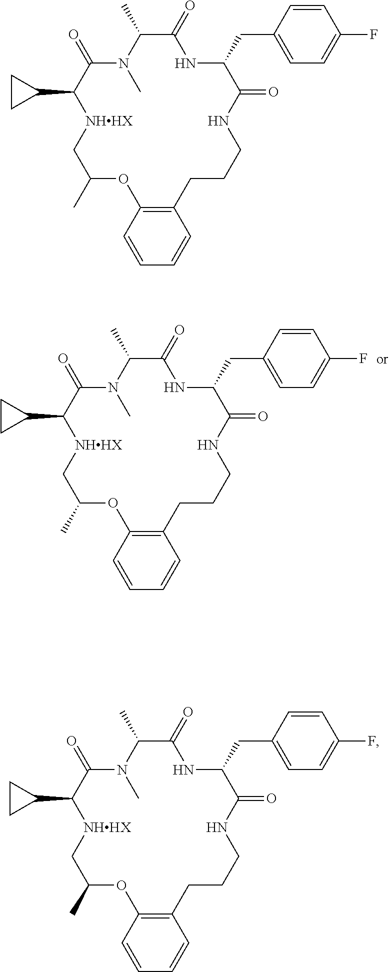
wherein HX is selected from the group consisting of carbonic acid, sulfuric acid, nitric acid, phosphoric acid, formic acid, acetic acid, propionic acid, maleic acid, succinic acid, mandelic acid, fumaric acid, malonic acid, citric acid, pyruvic acid, oxalic acid, stearic acid, ascorbic acid, glycolic acid, salicylic acid, a pyranosidyl acid, an alpha-hydroxy acid, an amino acid, an aromatic acid and a sulfonic acid.

27. The salt of claim 26, wherein the acid is succinic acid, malonic acid, maleic acid, fumaric acid, malic acid, tartaric acid, citric acid, lactic acid, formic acid, sulfuric acid, phosphoric acid, methylsulfonic acid or ethylsulfonic acid.

28. The salt of claim 26, wherein the salt is an amorphous form.

29. The salt of claim 26, wherein the salt is a crystalline form.

30. A pharmaceutical composition comprising:
(a) a salt of claim 26; and
(b) a pharmaceutically acceptable carrier, excipient or diluent.

31. A polymorphic form of a salt of a macrocyclic compound with the following structure: 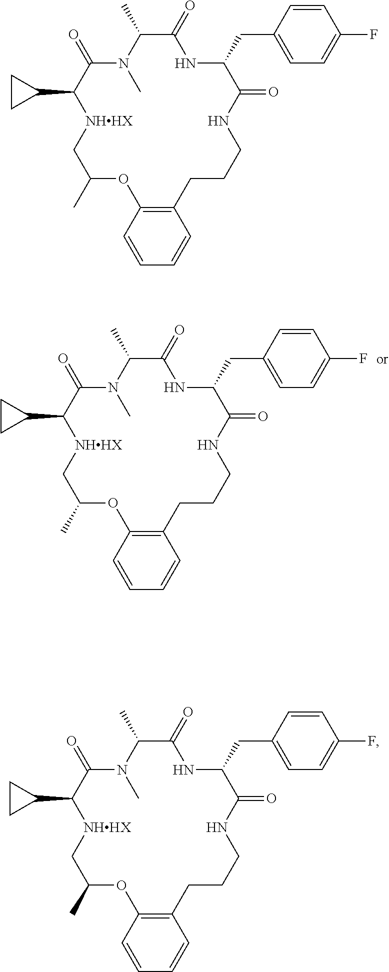
wherein HX is selected from the group consisting of hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, a pyranosidyl acid, an alpha-hydroxy acid, an amino acid, an aromatic acid and a sulfonic acid.

32. A pharmaceutical composition comprising:
(a) a polymorphic form of claim 31; and
(b) a pharmaceutically acceptable carrier, excipient or diluent.

33. A process for preparing a pharmaceutical composition of claim 17 comprising the following steps:
(a) dissolving a tonicity agent in solvent to form solution D;
(b) adding acid to solution D to form acidic solution D;
(c) dissolving a macrocyclic compound with the structure 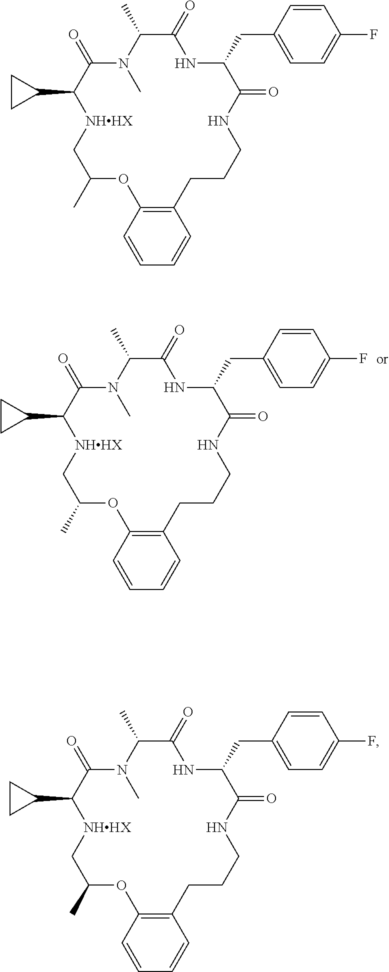
wherein HX is selected from the group consisting of hydrochloric acid, hydrobromic acid, hydroiodic acid, carbonic acid, sulfuric acid, nitric acid, phosphoric acid, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, a pyranosidyl acid, an alpha-hydroxy acid, an amino acid, an aromatic acid and a sulfonic acid; in acidified solution D to form solution E;
(d) adjusting the pH of solution E through the addition of base to form solution F; and
(e) diluting solution F with solvent to an effective concentration.

34. The process of claim 33, wherein the steps are conducted in the order step (b), then step (d), then step (a), then step (c), then step (e).

35. The process of claim 33, wherein the tonicity agent is dextrose.

36. The process of claim 33, wherein the acid is acetic acid.

37. The process of claim 33, wherein the base is sodium hydroxide.

38. The process of claim 33, further comprising filtration though one or more sterilizing filters.

39. A method of stimulating gastrointestinal motility by treating a subject with a pharmaceutical composition of claim 17, 24, 30 or 32.

40. The method of claim 39, wherein the pharmaceutical composition is administered parenterally or intravenously.

41. The method of claim 40, wherein the intravenous administration is via infusion.

42. The method of claim 39, wherein the pharmaceutical composition is administered subcutaneously.

43. The method of claim 39, wherein the pharmaceutical composition is administered orally.

44. The method of claim 39, wherein the subject is a human.

45. The method of claim 39, wherein the subject is a horse.

46. A method of treating a gastrointestinal disorder by administering to a subject a pharmaceutical composition of claim 17, 24, 30 or 32.

47. The method of claim 46, wherein the gastrointestinal disorder is selected from the group consisting of postoperative ileus, gastroparesis, opioid-induced bowel dysfunction, chronic intestinal pseudo-obstruction, acute colonic pseudo-obstruction (Ogilvie's syndrome), enteric dysmotility, short bowel syndrome, emesis, constipation-predominant irritable bowel syndrome (IBS), chronic constipation, functional dyspepsia, cancer-associated dyspepsia syndrome, graft versus host disease, delayed gastric emptying, gastrointestinal dysfunction or delayed gastric emptying occurring in conjunction with other disease states, gastrointestinal dysmotility or delayed gastric emptying occurring in critical care situations, gastrointestinal dysfunction or delayed gastric emptying as a result of treatment with pharmaceutical agents, gastroesophageal reflux disease (GERD), gastric ulcers, gastroenteritis and Crohn's disease.

48. The method of claim 46, wherein the gastrointestinal disorder is postoperative ileus.

49. The method of claim 46, wherein the gastrointestinal disorder is gastroparesis.

50. The method of claim 50, wherein the gastroparesis is diabetic gastroparesis or postsurgical gastroparesis.

51. The method of claim 47, wherein the gastrointestinal dysfunction or delayed gastric emptying occurs in patients with Parkinson's disease, myotonic muscular dystrophy, scerloderma, critical illness, eating disorders, autonomic degeneration, stroke, multiple sclerosis, neurological diseases, psychiatric diseases, cystic fibrosis, connective tissue diseases, cirrhosis, liver failure, renal failure, gallbladder disorders, migraines, brain stem lesions, spinal cord injury, cancer, neoplasia, achalasia, infectious diseases, Turner's syndrome, endocrine, metabolic or electrolyte disturbances, trauma or pain.

52. The method of claim 47, wherein the gastrointestinal dysfunction or delayed gastric emptying occurs as a result of treatment with opioids, anticholinergics, beta blockers, calcium channel antagonists, glucagon-like peptide-1 (GLP-1) receptor agonists, amylin receptor agonists, peptide YY (PYY) receptor agonists, proteasome inhibitors, tricyclic antidepressants, monoamine uptake blocker antidepressants, cancer chemotherapy agents, adrenergic agonists, dopaminergic agents, antimalarials, antispasmodics, cannabinoid agonists, octreotide, levodopa, alcohol or nicotine.

53. The method of claim 46, wherein the subject is a human.

54. The method of claim 46, wherein the subject is a horse.

55. A method of treating a subject suffering from a disorder characterized by lack of appetite, suppressed appetite, or that results in decreased food intake comprising administering to a subject in need thereof a pharmaceutical composition of claim 17, 24, 30 or 32.

56. The method of claim 55, wherein the disorder is cachexia.

57. The method of claim 55, wherein the cachexia is induced by cancer, chronic heart failure, acquired immunodeficiency syndrome (AIDS), renal disease, muscular dystrophies or aging.

58. The method of claim 55, wherein the subject is a human.

59. A method of treating a subject suffering from a metabolic and/or endocrine disorder, cardiovascular disorder, central nervous system disorder, bone disorder, inflammatory disorder, hyperproliferative disorder, disorder characterized by apoptosis or genetic disorder comprising administering to a subject in need thereof a pharmaceutical composition of claim 17, 24, 30 or 32.

60. A kit containing a pharmaceutical composition of claim 17, 24, 30 or 32.

61. The kit of claim 60, wherein the pharmaceutical composition is contained in vials or syringes.

62. The pharmaceutical composition of any of claim 17, 24, 30 or 32, wherein the pharmaceutical composition comprises the salt, solvate or polymorphic form in an amount in a range from about 75% to about 99.9%.
